(19) United States
(12) Patent Application Publication
(10) Pub. No.: US 2014/0322278 A1
(43) Pub. Date: Oct. 30, 2014

(54) Modulation of Tissue Transglutaminase Activation in Disease

(71) Applicants: Thomas DiRaimondo, Orinda, CA (US); Xi Jin, Stanford, CA (US); Cornelius Kloeck, Stanford, CA (US); Chaitan Khosla, Palo Alto, CA (US)

(72) Inventors: Thomas DiRaimondo, Orinda, CA (US); Xi Jin, Stanford, CA (US); Cornelius Kloeck, Stanford, CA (US); Chaitan Khosla, Palo Alto, CA (US)

(73) Assignee: The Board of Trustees of the Leland Stanford Junior University, Palo Alto, CA (US)

(21) Appl. No.: 14/126,629

(22) PCT Filed: Jun. 19, 2012
(86) PCT No.: PCT/US12/43150

§ 371 Date: May 7, 2014

Related U.S. Application Data
(60) Provisional application No. 61/499,044, filed on Jun. 20, 2011.

(51) Int. Cl. A61K 31/5377; A61K 31/428; A61K 31/423; A61K 31/4184

(52) U.S. Cl. CPC A61K 31/5377; A61K 31/4184; A61K 31/428; A61K 31/423
USPC 424/400; 514/233.5; 544/151; 514/394; 548/310.1; 514/367; 548/179; 514/375; 548/217

(57) ABSTRACT

Compositions and methods are provided for modulating the physiological activation of tissue transglutaminase (TG2); which methods can include inhibiting the activation of TG2 associated with enteric inflammatory disorders, which disorders may include celiac disease, irritable bowel syndrome, Crohn's Disease, dermatitis herpetiformis, and the like. In other embodiments of the invention, methods are provided for reducing undesirable paracellular transport in enteric tissues, in particular the paracellular transport of molecules greater than about 500 mw, e.g. peptides, including without limitation immunogenic gluten peptides.

### GOVERNMENT RIGHTS

This invention was made with Government support under contract DK063158 awarded by the National Institutes of Health. The Government has certain rights in this invention.

### BACKGROUND OF THE INVENTION

Celiac sprue (also known as celiac disease or coeliac disease) is a chronic inflammatory disease of the small intestine that occurs at a frequency of 0.5-1% in most populations around the world. The environmental trigger of celiac sprue is dietary gluten from common food grains such as wheat, rye, and barley. Duodenal digestion of gluten releases proteolytically resistant, immunotoxic peptide fragments, such as the immunodominant 33-mer from α-gliadin. These peptides are transported across the mucosal epithelial barrier and are deamidated at specific glutamine residues by an endogenous enzyme, transglutaminase 2 (TG2). Deamidated peptides bind with high affinity to the primary genetic determinant of celiac sprue, human leukocyte antigen (HLA) DQ2, a class II major histocompatibility complex (MHC) molecule found in >90% of diagnosed celiac sprue cases. The remaining cases are associated with HLA DQ8.

Upon encountering DQ2-gluten complexes on the surface of antigen presenting cells (APC), gluten-specific, DQ2-restricted CD4⁺ T cells are activated to induce a Th1 response comprising the secretion of pro-inflammatory cytokines, such as IFN-γ, and the recruitment of CD8⁺ intraepithelial lymphocytes, ultimately causing mucosal damage. Additionally, CD4⁺ T cells give help to a humoral immune response comprising production of both gluten-specific antibodies and TG2-specific autoantibodies.

In many affected individuals, this molecular pathogenesis is manifested symptomatically as nutrient malabsorption, wasting, and/or chronic diarrhea, and chronic inflammation caused by recurrent exposure to gluten is associated with the increased incidence of T cell lymphoma of the small intestine. Inflammation, antibody production, and clinical symptoms are gluten-dependent, such that strict adherence to a gluten-free diet causes remission, while reintroduction of dietary gluten causes relapse. However, a gluten-free diet is extremely difficult to maintain due to the ubiquity of gluten in human foods. Consequently, non-dietary therapies could substantially improve the health and quality of life of celiac sprue patients.

Orally administered gluten-specific proteases (i.e., glutenases) are an attractive strategy for treating celiac sprue. For example, ALV003 is a two-enzyme combination oral therapy undergoing clinical trials in celiac patients (ID#NCT01255696). Other treatment modalities are also being evaluated. For example, AT-1001 (ID#NCT00620451) is an investigational drug for celiac disease that is thought to reverse tight junction dysfunction in celiac patients, thereby preventing gluten transport across the epithelial layer. More recently, clinical trials have been initiated with Nexvax2, a prototypical vaccine based on a set of gluten peptides that are recognised by HLA-DQ2 in an immunodominant fashion (ID# NCT00879749).

Transglutaminases belong to a family of enzymes that play important roles in diverse biological functions by selectively cross-linking proteins. They catalyze formation of ε-(γ-glutamyl)-lysine cross-links between proteins, and may also incorporate polyamines into suitable protein substrates. This covalent isopeptide cross-link is stable and resistant to proteolysis, thereby increasing the resistance of tissue to chemical, enzymatic, and mechanical disruption. Among the members of this family are plasma transglutaminase, factor XIIIa, which stabilizes fibrin clots; keratinocyte transglutaminase and epidermal transglutaminase, which cross-link proteins on the outer surface of squamous epithelia; and tissue transglutaminase, which cross-links fibronectin in the extracellular matrix of organs such as brain, liver and the intestine.

Transglutaminase 2 (TG2, also known as tissue transglutaminase), a calcium-dependent member of the transglutaminase family, is reported to have extracellular as well as intracellular functions. Outside the cell, TG2 plays a crucial role in shaping the extracellular matrix by cross-linking fibronectin and related proteins. TG2 also promotes cell adhesion and motility by forming non-covalent complexes with other key proteins such as integrins and fibronectin. Intracellular TG2 loses enzyme activity when bound to GTP, but functions as a G-protein in the phospholipase C signal transduction cascade. Human TG2 is a structurally and mechanistically complex protein. Its catalytic mechanism is similar to that employed by cysteine proteases, involving a catalytic triad of cysteine, histidine, and aspartate. The cysteine thiol group reacts with a glutamine sidechain of a protein substrate to form a reactive thioester intermediate, from which the acyl group is transferred to another amine substrate.

Several members of the transglutaminase family have been linked to disease, including tissue transglutaminase (TG2), and the skin transglutaminases, TG1 and TG3. TG2 is a cytoplasmic enzyme present in many cells, including those in the blood vessel wall. Aberrant TG2 activity is believed to play a role in neurological disorders such as Alzheimer's, Parkinson's and Huntington's disease (see, for example, Kim et al. (2002) Neurochem. Int. 40:85-103; Karpuj et al. (2002) Nature Med. 8, 143-149). In Celiac Sprue, where TG2 is the predominant autoantigen, its pivotal role in unmasking antigenic epitopes by site specific deamidation of gluten peptides is well established.

Although a number of TG2 inhibitors have been used in biological studies over the past two decades, many of these compounds (e.g. monodansyl cadaverine) contain primary amines in addition to potential inhibitory motifs, and it remains unclear whether the observed effects are due to excess competing amines or by blockage of TG2 substrate turnover. A few studies have utilized a suicide inhibitor, L682777, which inhibits human TG2 (Lorand et al. (1998) Exp Eye Res. 66:531-6). However, L682777 was designed as a specific inhibitor of Factor XIIIa, and is therefore unsuitable for evaluating TG2 biology in vivo. More recently, mechanism-based active-site inhibitors of guinea pig and human (Hausch et al. (2003) Chem Biol 10, 225-231; Choi et al. (2005) Chem. Biol. 12, 469-475) TG2 have been reported.

In view of the serious and widespread nature of Celiac Sprue and the difficulty of removing gluten from the diet, better methods of treatment are of great interest. In particular, there is a need for treatment methods that allow the Celiac Sprue individual to eat gluten-containing foodstuffs without ill effect or at least to tolerate such foodstuffs in small or moderate quantities without inducing relapse.

### SUMMARY OF THE INVENTION

Compositions and methods are provided for modulating the physiological activation of tissue transglutaminase (TG2). Methods of the invention include inhibiting the activation of TG2 associated with enteric inflammatory disorders, which disorders may include celiac disease, irritable bowel syndrome, Crohn's Disease, dermatitis herpetiformis, and the like. The methods of the invention provide inhibitors that act in the molecular pathway involved in TG2 activation, including extracellular activation, during pathogenic processes. Included as target proteins for modulation are the antioxidant protein thioredoxin, and the isozymes of the phosphoinositide 3-kinase (PI3K) family. It is shown herein that activity of these proteins is required for in vivo activation of TG2, and that blocking the activity of one or both of these proteins inhibits TG2 activation in the local environment, and thereby blocks an essential step in the inflammatory response of disease-specific T cells to dietary gluten.

In some embodiments of the invention, an effective dose of an inhibitor of TG2 activation is administered to an individual suffering from an enteric inflammatory disorders, wherein the level of active TG2 in the individual, particularly the level of active enteric TG2, is decreased. In some embodiments of the invention, an inhibitor of a target protein described herein provides for appropriate safety characteristics associated with chronic inhibition of the target in the small intestine. In particular, inhibitors of interest have a high first pass metabolism and are active in the intestine. Inhibitors may be orally administered.

In other embodiments of the invention, methods are provided for reducing undesirable paracellular transport in enteric tissues, in particular the paracellular transport of molecules greater than about 500 mw, e.g. peptides, including without limitation immunogenic gluten peptides. Included as target proteins for modulation are the isozymes of the phosphoinositide 3-kinase (PI3K) family. Such undesirable paracellular transport may be associated with a variety of enteric disorders.

In other embodiments of the invention, assays are provided to identify candidate agents that act on TG2 activation, including high throughput in vitro cellular or cell-free assays. In the assays of the invention, TG2 is activated by enterocytes treated with γ-IFN. The level of TG2 activity can be monitored by methods known in the art, e.g. by determining the cross-linking of a TG2 substrate. The level of active enzyme may be compared to the total TG2 concentration, e.g. as determined by a suitable affinity assay, etc. Candidate agents may be brought in contact with the system, and the effect on TG2 activation determined after incubation for a period of time sufficient to measure activation where present. As controls, the assay may be compared to the activity on the absence of an agent, or in the presence of an agent shown herein to inhibit TG2 activation, e.g. inhibitors of PI3 kinase, inhibitors of thioredoxin, etc. Cell-free assays may utilize preparations of TG2 in the presence of thioredoxin and upon exposure to buffers with varying redox potentials, where the determination of TG2 activity is as described above. Candidate agents include, without limitation, inhibitors of γ-IFN, inhibitors of PI3 kinase, inhibitors of thioredoxin, inhibitors of TG2, and the like.

The invention also provides lead compounds and therapeutic agents that are inhibitors of thioredoxin, PI3K or TG2, as illustrated in Scheme 1. In some embodiments, the lead compound is a pharmacologically useful thioredoxin inhibitor, including PX12 (1) and analogs thereof. Examples of such compounds are set forth in Table 1 and Table 2 herein. In some embodiments the lead compound is a pharmacologically useful PI3K inhibitor, including LY294002 (2) and BEZ-235 (8) and analogs thereof. In some embodiments the lead compound is a pharmacologically useful TG2 inhibitor, including ERW1041E (3) and analogs thereof. Examples of such compounds are set forth in Table 3, Table 4 and Table 5 herein.

### BRIEF DESCRIPTION OF THE DRAWINGS

Scheme 1. Lead compounds against Thioredoxin (PX12, 1), PI3 Kinase (LY294002, 2) and Transglutaminase 2 (ERW1041E, 3).

Scheme 2. Other examples of promising PI3K inhibitors—compound 15e (4), TGX-221 (5), AS-252424 (6) and IC-87114 (7).

FIG. 1. T84 translocation assay used to measure flux of fluorescently labeled peptides and TG2 activation. (A) Transwell schematic illustrates transport of D8mer peptide control and antigenic gluten peptide, 33 mer. D_{P}′=paracellular mass flux of D8mer under IFN-γ condition. D_{P}=basal paracellular mass flux of D8mer. P_{T}′=transcellular mass flux of 33mer under IFN-γ condition. P_{T}=basal transcellular mass flux of 33mer. P_{P}′=paracellular mass flux of 33mer IFN-γ condition. P_{P}=basal paracellular mass flux of 33mer. If the dominant transport of 33mer uses the paracellular route then P_{P}′/P_{P}≈D_{P}′/D_{P}. Fluorescently labeled peptide molecular structures are also illustrated. (B) Peptide molar flux across T84 monolayers treated with IFN-γ for 48 h represented as average+/−standard deviation. Peptide flux reaches maximum when treated with at least 200 U/mL IFN-γ. (C) Average peptide flux normalized to 0 U/mL γ-IFN (basal) condition represented as mean+/−standard deviation.

FIG. 2. Dose and time dependence of TG2 activation in response to IFN-γ treatments of T84 monolayers measured in a quantitative enzyme linked immunosorbent type assay. T84 monolayers were treated for 1 to 72 hrs at 0-1000 U/mL IFN-γ with or without TG2 inhibition using 25 μM ERW1041E. TG2 activation was quantified by the amount of 5BP crosslinked to native proteins in the T84 cells as measured by tetramethylbenzidine turnover via streptavidin-HRP labeling. 5BP incorporation is dependent on IFN-γ exposure concentration. TG2 activation was highest at 1000 U/mL IFN-γ treatment and reduced to basal levels when TG2 was blocked using ERW1041E. 5BP incorporation was highest with the 72 hr IFN-γ incubation time and relatively little 5BP incorporation was seen in IFN-γ exposures less than 24 hr. Negative control wells incubated with 0 μM 5BP exhibited negligible signal. Data reported as mean+/−standard deviation.

FIG. 3. Dose dependence of PI3K inhibitor, LY294002, on the reduction of peptide permeability and TG2 activation in T84 models treated with various concentrations of IFN-γ. (A) LY294002 incubation reduces the increased Cy5-33mer and Cy3-D8mer permeability in T84 cells treated with IFN-γ for 48 h. At 10 μM, LY294002 reduces maximal peptide flux seen at the 1000 U/mL IFN-γ treatments to basal levels. DMSO levels were kept below 0.1% (v/v) in media. DMSO controls show no influence on peptide flux for any IFN-γ treatments tested. Data shown are normalized to 0 U/mL IFN-γ condition represented by mean+/−standard deviation. (B) Dose and time dependence of TG2 activation in response to IFN-γ treatments of T84 monolayers measured in a quantitative enzyme linked immunosorbent type assay. T84 monolayers were treated for 1 to 72 hrs at 0-1000 U/mL IFN-γ with or without PI3K inhibition using 10 μM LY294002. TG2 activation was quantified by the amount of 5BP crosslinked to native proteins in the T84 cells as measured by tetramethylbenzidine turnover via streptavidin-HRP labeling. Illustrates the dependence of 5BP incorporation on IFN-γ exposure concentration. TG2 activation was highest at 1000 U/mL IFN-γ treatment and reduced to basal levels when PI3K activity was blocked using LY294002. 5BP incorporation was highest with the 72 hr IFN-γ incubation time and relatively little 5BP incorporation was seen in IFN-γ exposures less than 24 hr. Negative control wells incubated with 0 μM 5BP exhibited negligible signal. Data reported as mean+/−standard deviation.

FIG. 4. Redox potential of the vicinal disulfide bond in oxTG2. (A) Time dependence of TG2 activity upon exposure to buffers with varying redox potentials. Oxidized TG2 (oxTG2) was pre-incubated for 1 h in buffers containing varying GSH/GSSG ratios, subject to a total [GSH]+[GSSG] concentration of 10 mM. Thereafter, TG2 activity was spectrophotometrically monitored in the presence of 20 mM ZQG substrate for 6 h at room temperature. (B) Steady-state enzyme activity as a function of redox potential. Specific activity was calculated based on 4-5 h slopes, and normalized to the activity of TG2 following 5 mM DTT treatment. (C) Steady state fraction of reduced TG2 as a function of redox potential. The fraction of reduced TG2 was determined based on the alkylation status of Cys370 and 371.

FIG. 5. Secretion of Trx by cultured T84 and THP-1 cells. Intracellular and extracellular Trx levels were quantified by western blot and ImageJ analysis. (A) Relative abundance of Trx on the apical (Top) and basolateral (Bottom) sides of cultured T84 monolayers that were treated with 1000 U/mL IFN-γ for 48 h. In both cases, Trx concentration is normalized to wells containing no IFN-γ. Because the medium volume on the basolateral side is twice that of the apical side volume, the rate of Trx secretion into the former volume is anticipated to be higher than the lateral volume. By way of comparison, the concentrations of intracellular Trx are also shown. In all cases, actin levels were used as a constant reference. (B) Relative abundance of Trx in the extracellular versus intracellular volumes of cultured THP-1 monocytic cells treated with 1000 U/mL IFN-γ for 48 h.

FIG. 6. Activation of TG2 in a co-culture comprising WI-38 fibroblasts and THP-1 cells. The two cell lines were co-cultured in 8-well glass chambers with or without 1000 U/mL IFN-γ for 48 h. The locations of intense TG2 activity (red) were visualized by 1 h 0.5 mM 5-BP incorporation following Alexa Fluor-555 staining, whereas the cells were visualized by phase contrast. All pictures are taken with 100× microscope.

FIG. 7. Dose dependence of TG2 activation in response to recombinant human thioredoxin treatments of T84 and WI-38 monolayers measured in a quantitative enzyme linked immunosorbent type assay. Cellular monolayers were treated for 3 hrs at 0-10 μM thioredoxin with or without TG2 inhibition using 25 μM ERW1041E or with or without using 0-50 μM thioredoxin inhibitor PX-12. TG2 activation was quantified by the amount of 5BP crosslinked to native proteins in the extracellular matrix of cultured monolayers as measured by tetramethylbenzidine turnover via streptavidin-HRP labeling. (A) and (B) Illustrate the dependence of 5BP incorporation on thioredoxin exposure concentration in T84 and WI-38 cells, respectively. TG2 activation was highest at 10 μM thioredoxin treatment and reduced to basal levels when TG2 was blocked using ERW1041E. (C) and (D) Illustrate the dependence of 5BP incorporation on the ability of thioredoxin to activate TG2. TG2 activation was highest for control experiments with 0 μM PX-12. The amount of PX-12 required to reduce the 5BP incorporation in half at saturating concentrations of 3 μM thioredoxin in T84 and WI-38 cultures was 11 μM and 5 μM PX-12, respectively. Negative control wells incubated with 0 μM 5BP exhibited negligible signal (data not shown). Data reported as mean+/−standard deviation.

FIG. 8. Effect of pan-PI3K inhibitor, BEZ235, on peptide permeability and TG2 activity in IFN-γ treated T84 monolayers. Permeability of (A) Cy3-D8mer across T84 monolayers treated with IFN-γ for 48 h. (B) TG2 activity, as measured by 5BP incorporation. (C) Structure of the pan PI3K inhibitor BEZ-235 (8). DMSO used to solubilize BEZ235 did not influence T84 permeability. DMSO levels were kept below 0.1% (v/v) in media. Data shown are normalized to 0 U/mL IFN-γ condition represented by mean+/−standard deviation.

### DETAILED DESCRIPTION

In celiac sprue, inflammation is triggered by disease-specific T cells that reside in the small intestine and recognize toxic gluten peptides from the diet. This recognition process is facilitated by modification of gluten peptides by TG2. As such, enteric inhibition of TG2 is generally regarded as a promising target for non-dietary therapy of celiac sprue. Under normal physiological conditions, extracellular TG2 is predominantly in an inactive form, and must be activated before gluten peptides can be deamidated. The mechanism by which TG2 is activated in the small intestine was previously unknown. The present invention provides an elucidation of the pathway for TG2 activation; and factors, such as the presence of thioredoxin, that modulate the activation.

This knowledge of the TG2 activation pathway has opened the possibility for design and use of candidate agents that inhibit such activation. Such agents find use in the treatment of conditions that include, without limitation, enteric inflammatory disorders, which disorders may include celiac disease, irritable bowel syndrome, Crohn's Disease, dermatitis herpetiformis, and the like. Included as target proteins for modulation are the antioxidant protein thioredoxin, and the isozymes of the phosphoinositide 3-kinase (PI3K) family. It is shown herein that activity of these proteins is required for in vivo activation of TG2, and that blocking the activity of one or both of these proteins inhibits TG2 activation in the local environment, and thereby blocks an essential step in the inflammatory response of disease-specific T cells to dietary gluten.

In some embodiments of the invention, an effective dose of an agent that blocks TG2 activation is administered to an individual suffering from undesirable TG2 activation, where the dose provides for a reduction in TG2 activity, particularly enteric TG2 activity. In some embodiments, the individual has been diagnosed with an inflammatory enteric disorder. In some embodiments the inflammatory enteric disorder is selected from celiac sprue, dermatitis herpetiformis, irritable bowel syndrome and Crohn's Disease. In some embodiments the agent inhibits PI3 kinase. In other embodiments the agent inhibits thioredoxin. In some embodiments the agent has a high first pass metabolism. In some embodiments the agent is administered orally and is active in the intestine. In some embodiments the agent is provided in a formulation with an enteric coating.

In other embodiments of the invention, methods are provided for reducing undesirable paracellular transport in enteric tissues, in particular the paracellular transport of molecules greater than about 500 mw, e.g. peptides, including without limitation immunogenic gluten peptides. Included as target proteins for modulation are the isozymes of the phosphoinositide 3-kinase (PI3K) family. Such undesirable paracellular transport may be associated with a variety of enteric disorders.

In some embodiments of the invention, an effective dose of an agent that inhibits intestinal paracellular transport is administered to an individual, where the dose provides for a reduction in paracellular transport of molecules larger than about 250 mw, usually larger than about 500 mw, or larger than about 1000 mw. In some embodiments, the individual has been diagnosed with an inflammatory enteric disorder. In some embodiments the inflammatory enteric disorder is selected from celiac sprue, dermatitis herpetiformis, irritable bowel syndrome and Crohn's Disease. In some embodiments the agent inhibits PI3 kinase. In some embodiments the agent has a high first pass metabolism. In some embodiments the agent is administered orally and is active in the intestine.

The therapeutic methods of the invention may be combined with therapies known in the art, including the administration of anti-inflammatory agents, administration of agents that directly inhibit TG2 activity, administration of glutenases, and the like, as known in the art.

In some embodiments of the invention, an inhibitor of a target protein described herein provides for appropriate safety characteristics associated with chronic inhibition of the target in the small intestine. In particular, inhibitors of interest have a high first pass metabolism and are active in the intestine. Inhibitors may be orally administered.

### DEFINITIONS

As used herein, the term “therapeutic drug” or “therapeutic regimen” refers to an agent used in the treatment or prevention of a disease or condition, particularly an enteropathic condition for the purposes of the present invention. Of interest are therapeutic treatment methods, clinical trials using such therapies, screening assays for such therapies, and monitoring of patients undergoing such therapy.

In some embodiments, the therapy involves treatment of an individual, e.g. an individual suffering from an inflammatory enteric condition, with an agent of the invention. Patients may be control patients that have not been treated, or patients subject to a clinical regimen of interest, e.g. dietary restriction of gluten, treatment with PI3K inhibitor, may be newly diagnosed, etc. A “patient,” or individual, as used herein, describes an organism, including mammals, particularly humans.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “pharmacokinetics,” refers to the mathematical characterization of interactions between normal physiological processes and a therapeutic drug over time (i.e., body effect on drug). Certain physiological processes (absorption, distribution, metabolism, and elimination) will affect the ability of a drug to provide a desired therapeutic effect in a patient. Knowledge of a drug's pharmacokinetics aids in interpreting drug blood stream concentration and is useful in determining pharmacologically effective drug dosages. As is known in the art, a high first pass metabolism renders a drug useful in localized areas and for short periods of time, but limits the systemic activity.

The term “in combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “isolated compound” means a compound which has been substantially separated from, or enriched relative to, other compounds with which it occurs in nature or during chemical synthesis. Isolated compounds are usually at least about 80% pure, or at least about 90% pure, at least about 98% pure, or at least about 99% pure, by weight. The present invention is meant to encompass diastereomers as well as their racemic and resolved, enantiomerically pure forms and pharmaceutically acceptable salts thereof.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is preferably sterile, and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Compounds included in the present compositions that are acidic in nature may react with any number of inorganic and organic bases to form pharmaceutically acceptable base salts. Bases may include, for example, the mineral bases, such as NaOH and KOH, but one of skill in the art would appreciate that other bases may also be used. See Ando et al., Remington: The Science and Practice of Pharmacy, 20th ed. 700-720 (Alfonso R. Gennaro ed.), 2000.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

In some embodiments, the pharmaceutically acceptable addition salts of the compounds described herein may also exist as various solvates, such as, for example, with water, methanol, ethanol, dimethylformamide, and the like. Mixtures of such solvates may also be prepared. The source of such solvate may be from the solvent of crystallization, inherent in the solvent of preparation or crystallization, or adventitious to such solvent.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

The terms “contact”, “contacts”, “contacting” have their normal meaning and refer to combining two or more entities (e.g., two proteins, a polynucleotide and a cell, a cell and a candidate agent, etc.) Contacting can occur in vitro, in situ or in vivo and is used interchangeably with “expose to”, “exposed to”, “exposing to.”

As used herein, the terms “reduce”, “decrease” and “inhibit” are used together because it is recognized that, in some cases, an observed activity can be reduced below the level of detection of a particular assay. As such, it may not always be clear whether the activity is “reduced” or “decreased” below a level of detection of an assay, or is completely “inhibited”.

As used herein, compounds which are “commercially available” may be obtained from standard commercial sources including Acros Organics (Geel Belgium), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.); Molecular Probes (Eugene, Oreg.); Invitrogen (Carlsbad, Calif.), Applied Biosystems, Inc. (Foster City, Calif.), Glen Research (Sterling, Va.), Biosearch Technologies (Novato, Calif.), Anaspec (Fremont, Calif.) and Berry & Associates (Dexter, Mich.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. The term lower alkyl will be used herein as known in the art to refer to an alkyl, straight, branched or cyclic, of from about 1 to 6 carbons. It will be understood by those skilled in the art, with respect to any group containing one or more substituents, that such groups are not intended to introduce any substitution or substitution patterns that are sterically impractical, synthetically non-feasible and/or inherently unstable.

When describing the compounds, pharmaceutical compositions containing such compounds and methods of using such compounds and compositions, the following terms have the following meanings unless otherwise indicated. It should also be understood that any of the moieties defined forth below may be unsubstituted or substituted with a variety of substituents, and that the respective definitions are intended to include both unsubstituted and substituted moieties within their scope.

“Acyl” refers to a —C(O)R group, where R is hydrogen, alkyl, alkenyl, cycloalkyl, heterocycloalkyl, aryl, arylalkyl, heteroalkyl, heteroalkenyl, or heteroaryl as defined herein. Representative examples include, but are not limited to, formyl, acetyl, cyclohexylcarbonyl, cyclohexylmethylcarbonyl, benzoyl, benzylcarbonyl and the like.

“Acylamino” refers to a —NR′C(O)R group, where R′ is hydrogen, alkyl, cycloalkyl, heterocycloalkyl, aryl, arylalkyl, heteroalkyl, heteroaryl, heteroarylalkyl and R is hydrogen, alkyl, alkoxy, cycloalkyl, heterocycloalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)H, —OC(O)-alkyl, —OC(O)-aryl or —OC(O)-cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkynyl and alkynylene. Lower aliphatic groups typically have from 1 or 2 to 6 or 12 carbon atoms.

“Alkenyl” refers to monovalent olefinically unsaturated hydrocarbyl groups having up to about 11 carbon atoms, such as from 2 to 8 carbon atoms, and including from 2 to 6 carbon atoms, which can be straight-chained or branched and having at least 1 and including from 1 to 2 sites of olefinic unsaturation. Particular alkenyl groups include ethenyl (—CH═CH2), n-propenyl (—CH2CH═CH2), isopropenyl (—C(CH3)═CH2), vinyl and substituted vinyl, and the like.

“Alkoxy” refers to the group —O-alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Alkoxycarbonylamino” refers to the group —NRC(O)OR′ where R is hydrogen, alkyl, aryl or cycloalkyl, and R′ is alkyl or cycloalkyl.

“Alkyl” refers to monovalent saturated aliphatic hydrocarbyl groups particularly having up to about 12 or 18 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Alkylene” refers to divalent saturated aliphatic hydrocarbyl groups particularly having up to about 12 or 18 carbon atoms and more particularly 1 to 6 carbon atoms which can be straight-chained or branched. This term is exemplified by groups such as methylene (—CH2-), ethylene (—CH2CH2-), the propylene isomers (e.g., —CH2CH2CH2- and —CH(CH3)CH2-) and the like.

“Alkynyl” refers to acetylenically unsaturated hydrocarbyl groups particularly having up to about 12 or 18 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH2C≡CH), and the like.

“Amino” refers to the radical —NH2.

“Aminocarbonyl” refers to the group —C(O)NRR where each R is independently hydrogen, alkyl, aryl or cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aralkyl” or “arylalkyl” refers to an alkyl group, as defined above, substituted with one or more aryl groups, as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some cases, an aryl group includes from 6 to 14 carbon atoms.

“Aryloxy” refers to —O-aryl groups wherein “aryl” is as defined herein.

“Azido” refers to a —N3 group.

“Carbonyl” refers to —C(O)— groups, for example, a carboxy, an amido, an ester, a ketone, or an acyl substituent.

“Carboxyl” refers to a —C(O)OH group

“Cyano” refers to a —CN group.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl.

“Halogen” or “halo” refers to fluoro, chloro, bromo and iodo.

“Hetero” when used to describe a compound or a group present on a compound means that one or more carbon atoms in the compound or group have been replaced by, for example, a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g., heterocycloalkenyl, cycloheteroalkenyl, e.g., heterocycloheteroalkenyl and the like having from 1 to 5, and particularly from 1 to 3 heteroatoms. A heteroatom is any atom other than carbon or hydrogen and is typically, but not exclusively, nitrogen, oxygen, sulfur, phosphorus, boron, chlorine, bromine, or iodine.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. The heteroaryl group can be a 5-20 membered heteroaryl, or 5-10 membered heteroaryl. Particular heteroaryl groups are those derived from thiophen, pyrrole, benzothiophene, benzofuran, indole, pyridine, quinoline, imidazole, oxazole and pyrazine.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings include from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C1 to C4 alkoxy group, similarly, “lower alkylthio” means a C1 to C4 alkylthio group.

“Heterocycle” refers to organic compounds that contain a ring structure containing atoms in addition to carbon, such as sulfur, oxygen or nitrogen, as part of the ring. They may be either simple aromatic rings or non-aromatic rings. Examples include azoles, morpholine, piperazine, pyridine, pyrimidine and dioxane. The maximum number of heteroatoms in a stable, chemically feasible heterocyclic ring, whether it is aromatic or non-aromatic, is determined by factors such as, the size of the ring, the degree of unsaturation and the valence of the heteroatoms. In general, a heterocyclic ring may have one to four heteroatoms so long as the heteroaromatic ring is chemically feasible and stable.

“Hydroxyl” refers to a —OH group.

“Stereoisomer” as it relates to a given compound refers to another compound having the same molecular formula, wherein the atoms making up the other compound differ in the way they are oriented in space, but wherein the atoms in the other compound are like the atoms in the given compound with respect to which atoms are joined to which other atoms (e.g. an enantiomer, a diastereomer, or a geometric isomer). See for example, Morrison and Boyd, Organic Chemistry, 1983, 4th ed., Allyn and Bacon, Inc., Boston, Mass., p. 123.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of acyl, acylamino, acyloxy, alkoxy, alkoxycarbonyl, alkoxycarbonylamino, amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, thioaryloxy, thioketo, thiol, alkyl-S(O)—, aryl-S(O)—, alkyl-S(O)₂— and aryl-S(O)₂. Substituents of interest may include, but are not limited to, —X, —R8 (with the proviso that R8 is not hydrogen), —O—, ═O, —OR8, —SR8, —S—, ═S, —NR8R9, ═NR8, —CX3, —CF3, —CN, —OCN, —SCN, —NO, —NO2, ═N2, —N3, —S(O)2O—, —S(O)2OH, —S(O)2R8, —OS(O2)O—, —OS(O)2R8, —P(O)(O—)2, —P(O)(OR8)(O—), —OP(O)(OR8)(OR9), —C(O)R8, —C(S)R8, —C(O)OR8, —C(O)NR8R9, —C(O)O—, —C(S)OR8, —NR10C(O)NR8R9, vNR10C(S)NR8R9, —NR11C(NR10)NR8R9 and —C(NR10)NR8R9, where each X is independently a halogen and R8 is an alkyl, an alkenyl, an alkynyl, a heterocycle or an aryl.

“Sulfonyl” refers to the group —SO2-. Sulfonyl includes, for example, methyl-SO2-, phenyl-SO2-, and alkylamino-SO2-.

“Sulfinyl” refers to the group —S(O)—.

“Thioalkoxy” refers to the group —S-alkyl.

“Thioaryloxy” refers to the group —S-aryl.

“Thioketo” refers to the group ═S.

“Thiol” refers to the group —SH.

“Thio” refers to the group —S—. Thio includes, for example, thioalkoxy, thioaryloxy, thioketo and thiol.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

PI3 Kinase.

Phosphatidylinositol 3,4,5 triphosphate [Ptdlns(3,4,5)P₃]. [Ptdlns(3,4,5)P₃] acts on pathways that control cell proliferation, cell survival and metabolic changes—often through protein kinases. This lipid can be produced by PI3 kinases, a family of related proteins (Van haesebroeck et al. (1997) TIBS 22:267; Toker and Cantley (1997) Nature 387:673676). Phosphatidylinositol 3-kinase (EC 2.7.1.137) is composed of 85-kD and 110-kD subunits. The 85-kD subunit lacks PI3-kinase activity and acts as an adapter, coupling the 110-kD subunit (p110) to activated protein tyrosine kinases. p110 may require a complex with p85-alpha for catalytic activity. The genetic and amino acid sequence of p110 subunits for human PI(3) kinase can be obtained from Genbank, accession numbers Z29090, X83368.

Agents of interest include inhibitors of PI(3) kinase, e.g. BEZ-235, wortmannin, LY294002, etc. and also include the compounds shown in scheme 2 herein. Physiologically effective levels of wortmannin range from about 10 to 1000 nM, usually from about 100 to 500 nM, and optimally at about 200 nM. Physiologically effective levels of LY294002 range from about 1 to 500 μM, usually from about 25 to 100 μM, and optimally at about 50 μM. The inhibitors are administered in vivo or in vitro at a dose sufficient to provide for these concentrations in the target tissue. Other inhibitors of PI(3) kinase include anti-sense reagents or siRNA that are specific for PI(3) kinase. Of particular interest are anti-sense molecules derived from the human PI(3) kinase sequence, particularly the catalytic p110 subunit, using the publicly available sequence. Alternatively, antibodies, antibody fragments and analogs or other blocking agents are used to bind to the PI(3) kinase in order to reduce the activity.

Thioredoxin is a 12-kD oxidoreductase enzyme containing a dithiol-disulfide active site. It is ubiquitous and found in many organisms from plants and bacteria to mammals. Multiple in vitro substrates for thioredoxin have been identified, including ribonuclease, choriogonadotropins, coagulation factors, glucocorticoid receptor, and insulin. Thioredoxins are characterized at the level of their amino acid sequence by the presence of two vicinal cysteines in a CXXC motif. These two cysteines are the key to the ability of thioredoxin to reduce other proteins. Thioredoxin proteins also have a characteristic tertiary structure termed the thioredoxin fold.

The thioredoxins are kept in the reduced state by the flavoenzyme thioredoxin reductase, in a NADPH-dependent reaction. Thioredoxins act as electron donors to peroxidases and ribonucleotide reductase. The related glutaredoxins share many of the functions of thioredoxins, but are reduced by glutathione rather than a specific reductase.

A number of inhibitors that target either Trx or TrxR to induce apoptosis have been described. For example, suberoylanilide hydroxamic acid (SAHA) functions by up-regulating an endogenous inhibitor of Trx. Other compounds target the selenocysteine-containing active site of TrxR. These include gold compounds, platinum compounds, arsenic trioxide, motexafin gadolinium, nitrous compounds, and various flavonoids. In addition, some compounds also convert TrxR to a ROS generating enzyme. PX-12 is currently in clinical trials as a thioredoxin inhibitor.

### Compositions and Methods of Use

Provided herein are therapeutic compounds that may be used to inhibit the activity of TG2, particularly enteric TG2. These compounds can be incorporated into a variety of formulations for therapeutic administration by a variety of routes. More particularly, the compounds disclosed herein can be formulated into pharmaceutical compositions by combination with appropriate, pharmaceutically acceptable carriers, diluents, excipients and/or adjuvants. The following are examples of compounds of the invention.

In certain embodiments, the subject compounds include a substituent that contributes to optical isomerism and/or stereo isomerism of a compound. Salts, solvates, hydrates, and prodrug forms of a compound are also of interest. All such forms are embraced by the present invention. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Compositions of interest include the thioredoxin inhibitor PX12 (1) and analogs thereof, including those compounds set forth in Table 1 below.

TABLE 1

| | | | | Trx | | |
|---|---|---|---|---|---|---|
| | | | | kᵢₙₕ/Kᵢ | DTT k | |
| | | Wavelength | Δε (M⁻¹ | (μM⁻¹ | (μM⁻¹ | |
| # | Structure | (nm) | cm⁻¹) | min⁻¹) | min⁻¹) | Trx/DTT |
| 1 | 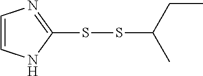 | 252 | 9400 | 0.16 | 0.0108 | 15 |
| 3 | 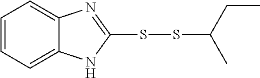 | 300 | 12064 | 0.19 | 0.0048 | 39 |
| 4 | 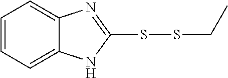 | 300 | 20630 | — | 0.054 | — |
| 5 | 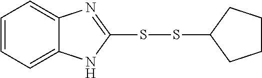 [SMILES: c1ccc2[nH]c(SSC3CCCC3)nc2c1] | 300 | 22708 | 0.24 | 0.0032 | 74 |
| 6 | 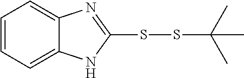 | 300 | 1370 | ~2.8 × 10⁻⁴ | ~2.8 × 10⁻⁵ | 10 |
| 7 | 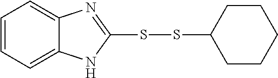 [SMILES: c1ccc2[nH]c(SSC3CCCCC3)nc2c1] | 300 | 25200 | 0.38 | 0.0046 | 83 |
| 8 | 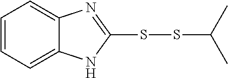 | 300 | 28620 | 0.33 | 0.0046 | 72 |
| 9 | 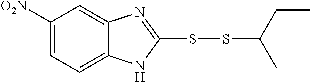 [SMILES: CCC(C)SSc1nc2cc([N+](=O)[O-])ccc2[nH]1] | 273 | 17194 | 0.87 | 0.0024 | 362 |
| 10 | 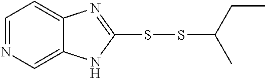 | 287 | −6650 | 0.19 | 0.0022 | 85 |
| 11 | 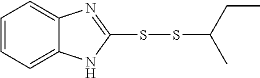 | 320 | 22548 | 0.50 | 0.0013 | 385 |
| 12 | 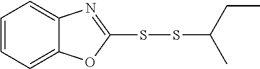 | 294 | 19104 | 2.4 | 0.0061 | 392 |
| 13 | 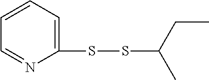 | 343 | 8732 | inactive | 3.7 × 10⁻⁵ | N/A |
| 14 | 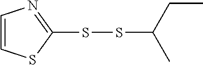 | 310 | 5600 | 0.050 | 0.00074 | 68 |
| 15 | 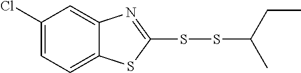 [SMILES: CCC(C)SSc1nc2cc(Cl)ccc2s1] | 325 | 15160 | 0.015 | 0.00017 | 88 |
| 16 | 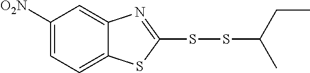 [SMILES: CCC(C)SSc1nc2cc([N+](=O)[O-])ccc2s1] | 372 | 3440 | 0.35 | 0.0020 | 175 |
| 17 | 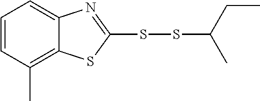 [SMILES: CCC(C)SSc1nc2cccc(C)c2s1] | 314 | 16100 | 0.049 | 0.00060 | 82 |

Physical properties and kinetic parameters of human thioredoxin inhibitors. The compounds below are analogs of PX-12 (1). Their
potency is estimated by the bimolecular parameter Kᵢₙₕ/Kᵢ. Their selectivity for human thioredoxin is estimated by the ratio of their
reactivity towards Trx relative to dithiothreitol (DTT).

Of particular interest are compounds:

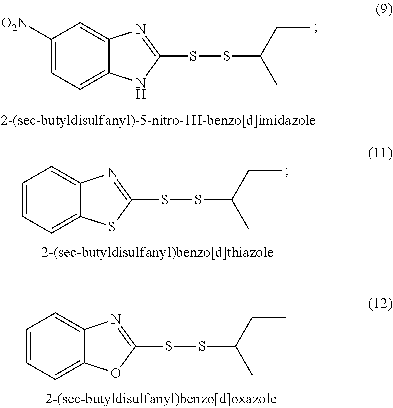 [SMILES: CCC(C)SSc1nc2cc([N+](=O)[O-])ccc2[nH]1 ; CCC(C)SSc1nc2ccccc2s1 ; CCC(C)SSc1nc2ccccc2o1]

which show more than 300-fold improvement in specificity.
Compounds also of interest include:

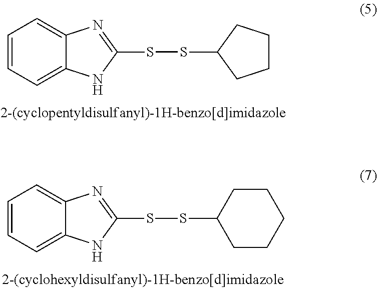 [SMILES: c1ccc2[nH]c(SSC3CCCC3)nc2c1 ; c1ccc2[nH]c(SSC3CCCCC3)nc2c1]

are also of particular interest, in which the volatile 2-butanethiol moiety is replaced, without penalty, by less volatile thiols. The 2-butanethiol leaving group was a source of dose-limiting toxicity of PX-12 in previous human clinical studies.

TABLE 2
¹H NMR (300 MHz, DMSO) of disulfide inhibitors

| # | Structure | ¹H NMR | Appearance |
|---|---|---|---|
| 1 | 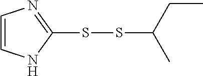 | δ 0.87, t, 3H; 1.29, d, 3H; 1.58, m, 2H; 3.04, m, 1H; 7.02, s, 1H; 7.29, s, 1H | white powder |
| 3 | 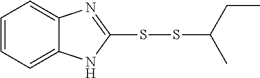 | δ 0.98, t, 3H; 1.32,d, 3H; 1.62, m, 2H; 2.99, m, 1H; 7.41, m, 1H; 7.67, m, 1H; 9.61, s, 1H | white powder |
| 4 | 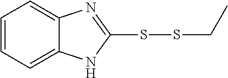 | δ 1.37, t, 3H; 2.88, m, 2H; 7.42, m, 1H; 7.68, m, 1H; 9.78, s, 1H | white powder |
| 5 | 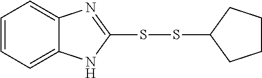 [SMILES: c1ccc2[nH]c(SSC3CCCC3)nc2c1] | δ 1.74, m, 4H; 2.01, m, 2H; 3.45, m, 1H; 7.42, s, 1H; 7.68; s, 1H; 9.68, s, 1H | white powder |
| 6 | 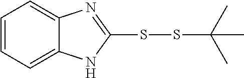 | δ 1.39, s, 9H; 7.50, m, 2H | white powder |
| 7 | 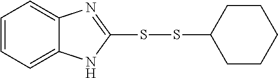 [SMILES: c1ccc2[nH]c(SSC3CCCCC3)nc2c1] | δ 1.32, m, 6H; 1.78, m, 2H; 2.03, m, 2H; 3.00, m, 1H; 7.24, d, 2H; 7.51, s, 2H | white powder |
| 8 | 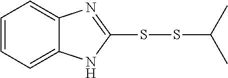 | δ 1.34, d, 6H; 3.25, m, 1H; 7.24, d, 2H; 7.51, s, 2H | white powder |
| 9 | 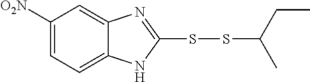 [SMILES: CCC(C)SSc1nc2cc([N+](=O)[O-])ccc2[nH]1] | δ 0.96, t, 3H; 1.33, d, 3H; 1.66, m, 2H; 3.06, m, 1H; 7.63, d, 1H; 8.18, d, 1H; 8.42, s, 1H | yellow solid |
| 10 | 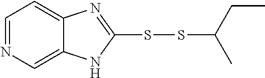 | δ 0.97, t, 3H; 1.32, d, 3H; 1.62, m, 2H; 3.04, m, 1H; 7.29, t, 1H; 7.92, d, 1H; 8.32, s, 1H | dark powder |
| 11 | 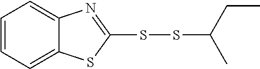 | δ 1.07, t, 3H; 1.38, d, 3H; 1.71, m, 2H; 3.10, m, 1H; 7.39, m, 2H; 7.85, m, 2H | light yellow liquid |
| 12 | 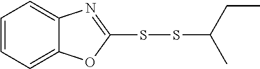 | δ 0.92, t, 3H; 1.29, d, 3H; 1.59, m, 2H; 3.18, m, 1H; 7.37, m, 2H; 7.71, m, 2H | dark liquid |
| 13 | 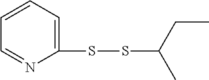 | δ 0.95, t, 3H; 1.24, d, 3H; 1.57, m, 2H; 2.98, m, 1H; 7.25, m, 1H; 7.76, m, 2H; 8.46, m, 1H | yellow liquid |
| 14 | 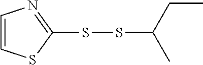 | δ 0.92, t, 3H; 1.31, d, 2H; 1.59, m, 2H; 3.11, m, 1H; 6.97, d, 1H; 7.26, d, 1H | light yellow liquid |
| 15 | 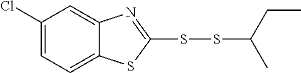 [SMILES: CCC(C)SSc1nc2cc(Cl)ccc2s1] | δ 0.98, t, 3H; 1.35, d, 3H; 1.64, m, 2H; 3.23, m, 1H; 7.47, m, 1H; 7.94, d, 1H; 8.10, d, 1H | white powder |
| 16 | 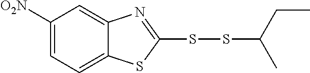 [SMILES: CCC(C)SSc1nc2cc([N+](=O)[O-])ccc2s1] | δ 0.99, t, 3H; 1.35, d, 3H; 1.68, m, 2H; 3.28, m, 1H; 8.01, d, 1H; 8.31, d, 1H; 9.12, s, 1H | yellow solid |
| 17 | 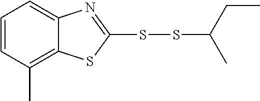 [SMILES: CCC(C)SSc1nc2cccc(C)c2s1] | δ 0.94, t, 3H; 1.29, d, 3H; 1.59, m, 2H; 2.90 s, 3H; 3.10, m, 1H; 7.27, s, 1H | light liquid |

Compositions of interest include the TG2 inhibitor ERW1041E (2) and analogs thereof, including those compounds set forth in Table 3 below.

TABLE 3

| | | kᵢₙₕ/Kᵢ | |
|---|---|---|---|
| | | [M⁻¹ min⁻¹] | |
| compound | ID | TG2 | TG1 |
| 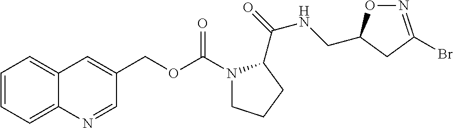 [SMILES: O=C(NC[C@@H]1CC(Br)=NO1)[C@@H]1CCCN1C(=O)OCc1cnc2ccccc2c1] | 2 | 16989 | 13222 |
| 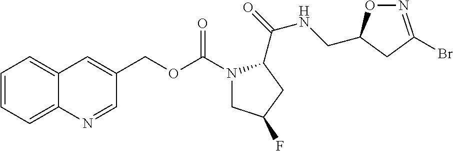 [SMILES: O=C(NC[C@@H]1CC(Br)=NO1)[C@@H]1C[C@@H](F)CN1C(=O)OCc1cnc2ccccc2c1] | 18 | 7250 | 6694 |
| 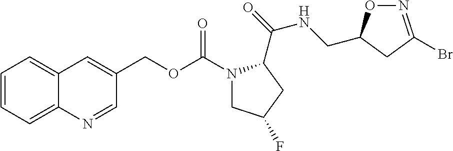 [SMILES: O=C(NC[C@@H]1CC(Br)=NO1)[C@@H]1C[C@H](F)CN1C(=O)OCc1cnc2ccccc2c1] | 19 | 13436 | 6040 |
| 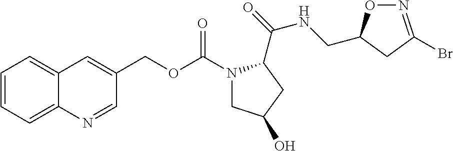 [SMILES: O=C(NC[C@@H]1CC(Br)=NO1)[C@@H]1C[C@@H](O)CN1C(=O)OCc1cnc2ccccc2c1] | 20 | 12511 | 4387 |
| 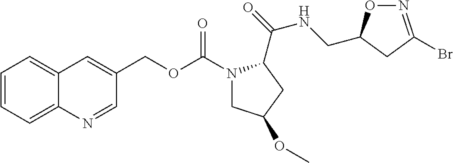 [SMILES: CO[C@@H]1C[C@@H](C(=O)NC[C@@H]2CC(Br)=NO2)N(C(=O)OCc2cnc3ccccc3c2)C1] | 21 | n/d | n/d |
| 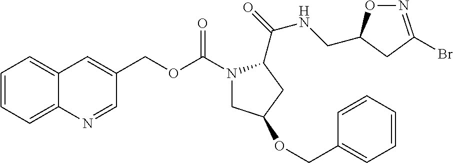 [SMILES: O=C(NC[C@@H]1CC(Br)=NO1)[C@@H]1C[C@@H](OCc2ccccc2)CN1C(=O)OCc1cnc2ccccc2c1] | 22 | 7710 | n/d |
| 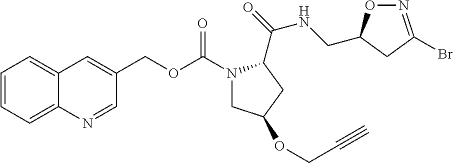 [SMILES: C#CCO[C@@H]1C[C@@H](C(=O)NC[C@@H]2CC(Br)=NO2)N(C(=O)OCc2cnc3ccccc3c2)C1] | 23 | 8518 | 4340 |

Analogs of ERW1041E (2). Although the parent compound has good activity towards human TG2, it was not
deemed satisfactory, because it showed comparable activity towards human transglutaminase 1 (TG1), and enzyme
essential for maintaining the structure and function of human skin.

Compounds of interest include, without limitation:

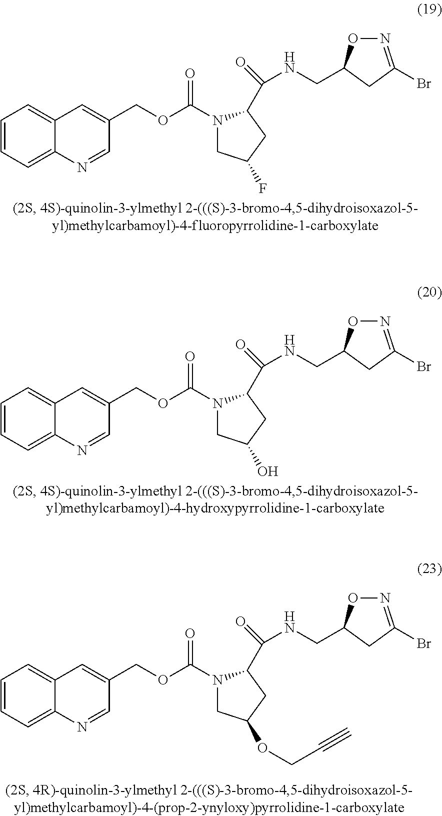 [SMILES: O=C(NC[C@@H]1CC(Br)=NO1)[C@@H]1C[C@H](F)CN1C(=O)OCc1cnc2ccccc2c1 ; O=C(NC[C@@H]1CC(Br)=NO1)[C@@H]1C[C@H](O)CN1C(=O)OCc1cnc2ccccc2c1 ; C#CCO[C@@H]1C[C@@H](C(=O)NC[C@@H]2CC(Br)=NO2)N(C(=O)OCc2cnc3ccccc3c2)C1]

which compounds have markedly improved specificity for TG2, and are therefore promising leads for therapeutic use. The last of these compounds is especially attractive due to the presence of an orthogonal alkyne group.

TABLE 4
Other ring-constrained analogs of ERW1041E (2).

| | | kᵢₙₕ/Kᵢ | |
|---|---|---|---|
| | | [M⁻¹ min⁻¹] | |
| compound | ID | TG2 | TG1 |
| 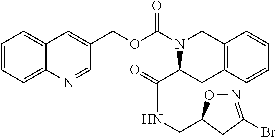 [SMILES: O=C(NC[C@@H]1CC(Br)=NO1)[C@@H]1Cc2ccccc2CN1C(=O)OCc1cnc2ccccc2c1] | 24 | 7800 | n/d |
| 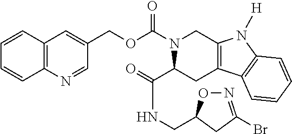 [SMILES: O=C(NC[C@@H]1CC(Br)=NO1)[C@@H]1Cc2c([nH]c3ccccc23)CN1C(=O)OCc1cnc2ccccc2c1] | 25 | 20800 | n/d |
| 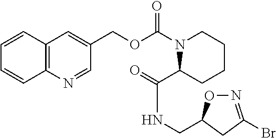 [SMILES: O=C(NC[C@@H]1CC(Br)=NO1)[C@@H]1CCCCN1C(=O)OCc1cnc2ccccc2c1] | 26 | 8297 | 23092 |

TABLE 5
¹H NMR of TG2 inhibitors

| compound | ID | |
|---|---|---|
| 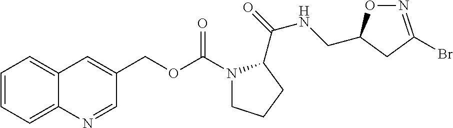 [SMILES: O=C(NC[C@@H]1CC(Br)=NO1)[C@@H]1CCCN1C(=O)OCc1cnc2ccccc2c1] | 2 | Published (Watts, Siegel and Khosla-J. Med. Chem. 2006, 49, 7493-7501.) |
| 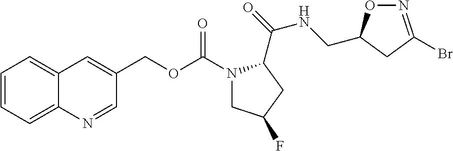 [SMILES: O=C(NC[C@@H]1CC(Br)=NO1)[C@@H]1C[C@@H](F)CN1C(=O)OCc1cnc2ccccc2c1] | 18 | ¹H NMR (400 MHz, CDCl₃) δ ppm: 8.86 (s, 1H); 8.08 (s, 1H); 8.06 (m, 1H); 7.76 (d, 1H); 7.61 (t, 1H); 7.49 (t, 1H); 7.16 (s, 1H); 5.40-5.10 (m, 3H); 4.70 (s, 1H); 4.40 (m, 1H); 4.00-3.80 (m, 1H); 3.70-2.90 (m, 5H); 2.50 (m, 1H); 2.30-2.10 (m, 1H). |
| 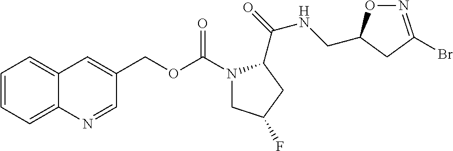 [SMILES: O=C(NC[C@@H]1CC(Br)=NO1)[C@@H]1C[C@H](F)CN1C(=O)OCc1cnc2ccccc2c1] | 19 | ¹H NMR (400 MHz, CDCl₃) δ ppm: 8.84 (s, 1H); 8.14 (s, 1H); 8.06 (d, 1H); 7.80 (d, 1H); 7.70 (t, 1H); 7.43 (t, 1H); 6.78 (s, 1H); 5.40-5.20 (m, 3H); 4.75 (s, 1H); 4.50 (s, 1H); 3.80 (m, 1H); 3.70-3.30 (m, 3H); 3.20-3.00 (m, 2H); 2.60 (m, 1H); 2.40-2.20 (m, 1H). |
| 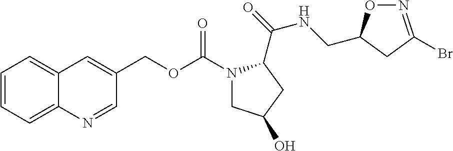 [SMILES: O=C(NC[C@@H]1CC(Br)=NO1)[C@@H]1C[C@@H](O)CN1C(=O)OCc1cnc2ccccc2c1] | 20 | ¹H NMR (400 MHz, CDCl₃) δ ppm: 8.87 (s, 1H); 8.11 (s, 1H); 8.08 (d, 1H); 7.79 (d, 1H); 7.69 (t, 1H); 7.53 (t, 1H); 7.04 (s, 1H); 5.30 (s, 1H); 5.23 (s, 1H); 4.76 (m, 1H); 4.45 (m, 2H); 3.75-3.60 (m, 3H); 3.38 (m, 1H); 3.20 (m, 1H); 3.02 (m, 1H); 2.35-2.15 (m, 2H). |
| 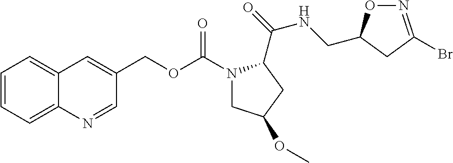 [SMILES: CO[C@@H]1C[C@@H](C(=O)NC[C@@H]2CC(Br)=NO2)N(C(=O)OCc2cnc3ccccc3c2)C1] | 21 | ¹H NMR (400 MHz, CDCl₃) δ ppm: 8.92 (s, 1H); 8.16 (d, 1H); 8.08 (m, 1H); 7.83 (d, 1H); 7.72 (t, 1H); 7.56 (t, 1H); 6.84 (s, 1H); 5.30 (m, 2H); 4.8 (m, 1H); 4.35 (t, 1H); 4.00 (s, 1H); 3.8-3.4 (m, 3H); 3.30 (s, 3H); 3.25 (m, 1H); 3.05 (m, 1H); 2.25 (m, 1H); 2.00 (m, 1H). |
| 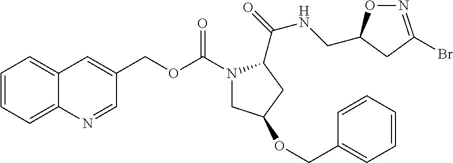 [SMILES: O=C(NC[C@@H]1CC(Br)=NO1)[C@@H]1C[C@@H](OCc2ccccc2)CN1C(=O)OCc1cnc2ccccc2c1] | 22 | ¹H NMR (400 MHz, CDCl₃) δ ppm: 8.93 (s, 1H); 8.15 (s, 1H); 8.12 (d, 1H); 7.82 (d, 1H); 7.74 (dt, 1H); 7.57 (dt, 1H); 7.4-7.2 (m, 5H); 5.34 (m, 2H); 4.79 (s, 2H); 4.46 (m, 2H); 4.2 (m, 1H); 3.69 (m, 1H); 3.58 (m, 1H); 3.49 (m, 1H); 3.22 (m, 1H); 3.05 (m, 1H); 2.31 (m, 1H); 1.94 (m, 1H) |
| 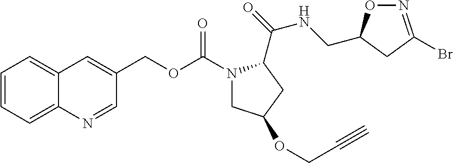 [SMILES: C#CCO[C@@H]1C[C@@H](C(=O)NC[C@@H]2CC(Br)=NO2)N(C(=O)OCc2cnc3ccccc3c2)C1] | 23 | ¹H NMR (400 MHz, CDCl₃) δ ppm: 8.89 (s, 1H); 8.1 (s, 1H); 8.06 (m, 1H); 7.80 (m, 1H); 7.68 (m, 1H); 7.52 (t, 1H); 6.90 (s, 1H); 5.30 (m, 2H); 4.75 (m, 1H); 4.30 (m, 1H); 4.10 (s, 3H); 3.8-3.5 (m, 2H); 3.40 (m, 1H); 3.20 (m, 1H); 3.00- 2.60 (m, 2H); 2.40 (m, 1H); 2.25 (m, 1H). |
| 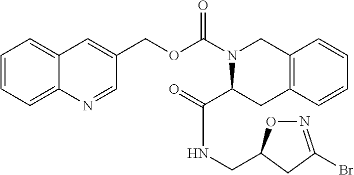 [SMILES: O=C(NC[C@@H]1CC(Br)=NO1)[C@@H]1Cc2ccccc2CN1C(=O)OCc1cnc2ccccc2c1] | 24 | ¹H NMR (400 MHz, DMSO-d₆) δ ppm: 8.91 (d, 1H); 8.17- 8.41 (m, 2H); 7.90-8.05 (m, 2H); 7.72-7.80 (m, 1H); 7.58- 7.66 (m, 1H); 7.10-7.25 (m, 4H); 5.20-5.41 (m, 2H); 4.38- 4.77 (m, 4H); 2.93-3.30 (m, 5H); 2.60-2.75 (m, 1H). |
| 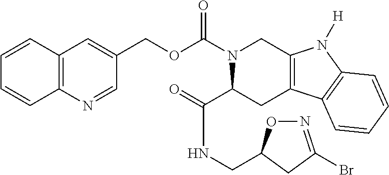 [SMILES: O=C(NC[C@@H]1CC(Br)=NO1)[C@@H]1Cc2c([nH]c3ccccc23)CN1C(=O)OCc1cnc2ccccc2c1] | 25 | ¹H NMR (400 MHz, DMSO-d₆) δ ppm: 10.81 (d, 1H); 8.97 (d, 1H); 8.32-8.44 (m, 2H); 7.97-8.07 (m, 2H); 7.78 (t, 1H); 7.64 (t, 1H); 7.37 (d, 1H); 7.28 (dd, 1H); 7.03 (t, 1H); 6.96 (t, 1H); 5.36-5.47 (m, 2H); 5.19 (t, 1H); 4.88 (t, 1H); 4.50-4.74 (m, 2H); 3.08-3.32 (m, 4H); 2.93-3.04 (m, 1H) 2.74-2.84 (m, 1H). |
| 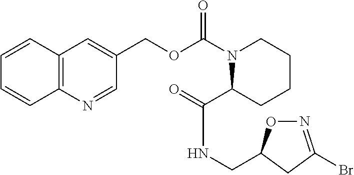 [SMILES: O=C(NC[C@@H]1CC(Br)=NO1)[C@@H]1CCCCN1C(=O)OCc1cnc2ccccc2c1] | 26 | ¹H NMR (400 MHz, CDCl₃) δ ppm: 8.90 (s, 1H); 8.14 (d, 1H); 8.09 (d, 1H); 7.82 (d, 1H); 7.71 (t, 1H); 7.55 (t, 1H); 6.48 (s, 1H); 5.33 (s, 2H); 4.77 (m, 2H); 3.52 (m, 1H); 3.45 (t, 1H); 3.23 (m, 1H); 3.00-2.85 (m, 2H); 1.5-1.7 (m, 4H). |

Agents that inhibit activation or activity of TG2 and/or that inhibit enteric paracellular transport are administered to an individual in need thereof, at a dose and for a period of time effective to achieve the desired result. The present invention provides the inhibitors in a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the inhibitors is achieved in various ways, although oral administration is a preferred route of administration. In some formulations, the inhibitor is localized by virtue of the formulation, such as the use of an implant that acts to retain the active dose at the site of implantation, or is otherwise localized by virtue of the relevant pharmacokinetics.

In some pharmaceutical dosage forms, the inhibitors are administered in the form of their pharmaceutically acceptable salts. In some dosage forms, the inhibitor is used alone, while in others, it is administered in combination with another pharmaceutically active compounds. In the latter embodiment, the other active compound is, in some embodiments, a glutenase that can cleave or otherwise degrade a toxic gluten oligopeptide.

For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

In one embodiment of the invention, the oral formulations comprise enteric coatings, so that the active agent is delivered to the intestinal tract. Enteric formulations are often used to protect an active ingredient from the strongly acid contents of the stomach. Such formulations are created by coating a solid dosage form with a film of a polymer that is insoluble in acid environments and soluble in basic environments. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate.

Other enteric formulations comprise engineered polymer microspheres made of biologically erodable polymers, which display strong adhesive interactions with gastrointestinal mucus and cellular linings, can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71(3):307-18. In another embodiment, the inhibitor or formulation thereof is admixed with food, or used to pre-treat foodstuffs containing glutens.

Formulations are typically provided in a unit dosage form, where the term “unit dosage form,” refers to physically discrete units suitable as unitary dosages for human subjects, each unit containing a predetermined quantity of inhibitor calculated in an amount sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the inhibitor is administered in dosages of 0.01 mg to 500 mg V/kg body weight per day, e.g. about 100 mg/day for an average person. Dosages are appropriately adjusted for pediatric formulation. Those of skill will readily appreciate that dose levels can vary as a function of the specific inhibitor, the diet of the patient and the gluten content of the diet, the severity of the symptoms, and the susceptibility of the subject to side effects. Some of the inhibitors of the invention are more potent than others. Preferred dosages for a given inhibitor are readily determinable by those of skill in the art by a variety of means. A preferred means is to measure the physiological potency of a given compound.

Various methods for administration are employed in the practice of the invention. In one preferred embodiment, oral administration, for example with meals, is employed. The dosage of the therapeutic formulation can vary widely, depending upon the nature of the disease, the frequency of administration, the manner of administration, the clearance of the agent from the patient, and the like. The initial dose can be larger, followed by smaller maintenance doses. The dose can be administered as infrequently as weekly or biweekly, or more often fractionated into smaller doses and administered daily, with meals, semi-weekly, and the like, to maintain an effective dosage level.

### Disease Conditions

Conditions of interest for methods of the present invention include a variety of enteropathic conditions, particularly chronic and inflammatory conditions. In some embodiments of the invention, a patient is diagnosed as having an enteropathic condition, for which treatment is contemplated. Enteropathic conditions of interest include, without limitation, Celiac Sprue, herpetiformis dermatitis, irritable bowel syndrome (IBS); and Crohn's Disease.

Celiac sprue is an immunologically mediated disease in genetically susceptible individuals caused by intolerance to gluten, resulting in mucosal inflammation, which causes malabsorption. Symptoms usually include diarrhea and abdominal discomfort. Onset is generally in childhood but may occur later. No typical presentation exists. Some patients are asymptomatic or only have signs of nutritional deficiency. Others have significant GI symptoms.

Celiac sprue can present in infancy and childhood after introduction of cereals into the diet. The child has failure to thrive, apathy, anorexia, pallor, generalized hypotonia, abdominal distention, and muscle wasting. Stools are soft, bulky, clay-colored, and offensive. Older children may present with anemia or failure to grow normally. In adults, lassitude, weakness, and anorexia are most common. Mild and intermittent diarrhea is sometimes the presenting symptom. Steatorrhea ranges from mild to severe (7 to 50 g fat/day). Some patients have weight loss, rarely enough to become underweight. Anemia, glossitis, angular stomatitis, and aphthous ulcers are usually seen in these patients. Manifestations of vitamin D and Ca deficiencies (eg, osteomalacia, osteopenia, osteoporosis) are common. Both men and women may have reduced fertility.

The diagnosis may be suspected clinically and by laboratory abnormalities suggestive of malabsorption. Family incidence is a valuable clue. Celiac sprue should be strongly considered in a patient with iron deficiency without obvious GI bleeding. Confirmation usually involves a small-bowel biopsy from the second portion of the duodenum. Findings include lack or shortening of villi (villous atrophy), increased intraepithelial cells, and crypt hyperplasia. Because biopsy results may be non-specific, serologic markers can aid diagnosis. Anti-gliadin antibody (AGA) and anti-endomysial antibody (EMA, an antibody against an intestinal connective tissue protein) in combination have a positive and negative predictive value of nearly 100%. These markers can also be used to screen populations with high prevalence of celiac sprue, including 1st-degree relatives of affected patients and patients with diseases that occur at a greater frequency in association with celiac sprue. If either test is positive, the patient may have a diagnostic small-bowel biopsy performed. If both are negative, celiac sprue is unlikely. Other laboratory abnormalities often occur and may be sought. These include anemia (iron-deficiency anemia in children and folate-deficiency anemia in adults); low albumin, Ca, K, and Na; and elevated alkaline phosphatase and PT. Malabsorption tests are sometimes performed, although they are not specific for celiac sprue. If performed, common findings include steatorrhea of 10 to 40 g/day and abnormal D-xylose and (in severe ileal disease) Schilling tests.

Conventional treatment is gluten-free diet (avoiding foods containing wheat, rye, or barley). Gluten is so widely used that a patient needs a detailed list of foods to avoid. Patients are encouraged to consult a dietitian and join a celiac support group. The response to a gluten-free diet is usually rapid, and symptoms resolve in 1 to 2 months. Ingesting even small amounts of food containing gluten may prevent remission or induce disease.

Complications include refractory sprue, collagenous sprue, and the development of intestinal lymphomas. Intestinal lymphomas affect 6 to 8% of patients with celiac sprue, usually presenting in the patient's 50s. The incidence of other GI malignancies (eg, carcinoma of the esophagus or oropharynx, small-bowel adenocarcinoma) increases. Adherence to a gluten-free diet can significantly reduce the risk of malignancy.

Dermatitis herpetiformis is a chronic eruption characterized by clusters of intensely pruritic vesicles, papules, and urticaria-like lesions. The cause is autoimmune. Diagnosis is by skin biopsy with direct immunofluorescence testing. Treatment is usually with dapsone or sulfapyridine.

This disease usually presents in patients 30 to 40 yr old and is rare in blacks and East Asians. It is an autoimmune disease. Celiac sprue is present in 75 to 90% of dermatitis herpetiformis patients and in some of their relatives, but it is asymptomatic in most cases. The incidence of thyroid disease is also increased. Iodides may exacerbate the disease, even when symptoms are well controlled. The term “herpetiformis” refers to the clustered appearance of the lesions rather than a relationship to herpesvirus.

Patients may have skin biopsy of a lesion and adjacent normal-appearing skin. IgA deposition in the dermal papillary tips is usually present and important for diagnosis. Patients should be evaluated for celiac sprue.

Strict adherence to a gluten-free diet for prolonged periods (eg, 6 to 12 mo) controls the disease in some patients, obviating or reducing the need for drug therapy. When drugs are needed, dapsone may provide symptomatic improvement. It is started at 50 mg po once/day, increased to bid or tid (or a once/day dose of 100 mg); this usually dramatically relieves symptoms, including itching, within 1 to 3 days; if so, that dose is continued. If no improvement occurs, the dose can be increased every week, up to 100 mg qid. Most patients can be maintained on 50 to 150 mg/day, and some require as little as 25 mg/wk. Although less effective, sulfapyridine may be used as an alternative for those who cannot tolerate dapsone. Initial oral dosage is 500 mg bid, increasing by 1 g/day q 1 to 2 wk until disease is controlled. Maintenance dosage varies from 500 mg twice/wk to 1000 mg once/day. Colchicine is another treatment option. Treatment continues until lesions resolve.

Crohn's Disease (Regional Enteritis; Granulomatous Ileitis or Ileocolitis) is a chronic transmural inflammatory disease that usually affects the distal ileum and colon but may occur in any part of the GI tract. Symptoms include diarrhea and abdominal pain. Abscesses, internal and external fistulas, and bowel obstruction may arise. Extraintestinal symptoms, particularly arthritis, may occur. Diagnosis is by colonoscopy and barium contrast studies. Treatment is with 5-aminosalicylic acid, corticosteroids, immunomodulators, anticytokines, antibiotics, and often surgery.

The most common initial presentation is chronic diarrhea with abdominal pain, fever, anorexia, and weight loss. The abdomen is tender, and a mass or fullness may be palpable. Gross rectal bleeding is unusual except in isolated colonic disease, which may manifest similarly to ulcerative colitis. Some patients present with an acute abdomen that simulates acute appendicitis or intestinal obstruction. About 33% of patients have perianal disease (especially fissures and fistulas), which is sometimes the most prominent or even initial complaint. In children, extraintestinal manifestations frequently predominate over GI symptoms; arthritis, fever of unknown origin, anemia, or growth retardation may be a presenting symptom, whereas abdominal pain or diarrhea may be absent.

With recurrent disease, symptoms vary. Pain is most common and occurs with both simple recurrence and abscess formation. Patients with severe flare-up or abscess are likely to have marked tenderness, guarding, rebound, and a general toxic appearance. Stenotic segments may cause bowel obstruction, with colicky pain, distention, obstipation, and vomiting. Adhesions from previous surgery also may produce bowel obstruction, which begins rapidly, without the prodrome of fever, pain, and malaise typical of obstruction due to a Crohn's disease flare-up. An enterovesical fistula may produce air bubbles in the urine (pneumaturia). Draining cutaneous fistulas may occur. Free perforation into the peritoneal cavity is unusual.

Crohn's disease should be suspected in a patient with inflammatory or obstructive symptoms or in a patient without prominent GI symptoms but with perianal fistulas or abscesses or with otherwise unexplained arthritis, erythema nodosum, fever, anemia, or (in a child) stunted growth. A family history of Crohn's disease also increases the index of suspicion. Patients presenting with an acute abdomen (either initially or on relapse) should have flat and upright abdominal x-rays and an abdominal CT scan. These studies demonstrate obstruction, abscesses or fistulas, and other possible causes of an acute abdomen (eg, appendicitis). Ultrasound may better delineate gynecologic pathology in women with lower abdominal and pelvic pain.

If initial presentation is less acute, an upper GI series with small-bowel follow-through and spot films of the terminal ileum is preferred over conventional CT. However, newer techniques of CT enterography, which combines high-resolution CT with large volumes of ingested contrast, are becoming the procedures of choice in some centers. These imaging studies are virtually diagnostic if they show characteristic strictures or fistulas with accompanying separation of bowel loops. If findings are questionable, CT enteroclysis or video capsule enteroscopy may show superficial aphthous and linear ulcers. Barium enema x-ray may be used if symptoms appear predominantly colonic (eg, diarrhea) and may show reflux of barium into the terminal ileum with irregularity, nodularity, stiffness, wall thickening, and a narrowed lumen. Differential diagnoses in patients with similar x-ray findings include cancer of the cecum, ileal carcinoid, lymphosarcoma, systemic vasculitis, radiation enteritis, ileocecal TB, and ameboma.

Established Crohn's disease is rarely cured but is characterized by intermittent exacerbations and remissions. Some patients suffer severe disease with frequent, debilitating periods of pain. However, with judicious medical therapy and, where appropriate, surgical therapy, most patients function well and adapt successfully. Disease-related mortality is very low. GI cancer, including cancer of the colon and small bowel, is the leading cause of excess Crohn's disease-related mortality.

Irritable bowel syndrome consists of recurring upper and lower GI symptoms, including variable degrees of abdominal pain, constipation or diarrhea, and abdominal bloating. Diagnosis is clinical. Treatment is generally symptomatic, consisting of dietary management and drugs, including anticholinergics and agents active at serotonin receptors.

There are no consistent motility abnormalities. Some patients have an abnormal gastro-colonic reflex, with delayed, prolonged colonic activity. There may be reduced gastric emptying or disordered jejunal motility. Some patients have no demonstrable abnormalities, and in those that do, the abnormalities may not correlate with symptoms. Small-bowel transit varies: sometimes the proximal small bowel appears to be hyperreactive to food or parasympathomimetic drugs. Intraluminal pressure studies of the sigmoid show that functional constipation can occur with hyperreactive haustral segmentation (ie, increased frequency and amplitude of contractions). In contrast, diarrhea is associated with diminished motor function. Thus strong contractions can, at times, accelerate or delay transit.

Hypersensitivity to normal amounts of intraluminal distention and heightened perception of pain in the presence of normal quantity of intestinal gas exist. Pain seems to be caused by abnormally strong contractions of the intestinal smooth muscle or by increased sensitivity of the intestine to distention. Hypersensitivity to the hormones gastrin and cholecystokinin may also be present. However, hormonal fluctuations do not correlate with symptoms. Meals of high caloric density may increase the magnitude and frequency of myoelectrical activity and gastric motility. Fat ingestion may cause a delayed peak of motor activity, which can be exaggerated in IBS. The first few days of menstruation can lead to transiently elevated prostaglandin E2, resulting in increased pain and diarrhea, probably by the release of prostaglandins.

Two major clinical types of IBS have been described. In constipation-predominant IBS, most patients have pain over at least one area of the colon and periods of constipation alternating with a more normal stool frequency. Stool often contains clear or white mucus. Pain is either colicky, coming in bouts, or a continuous dull ache; it may be relieved by a bowel movement. Eating commonly triggers symptoms. Bloating, flatulence, nausea, dyspepsia, and pyrosis can also occur.

Diarrhea-predominant IBS is characterized by precipitous diarrhea that occurs immediately on rising or during or immediately after eating, especially rapid eating. Nocturnal diarrhea is unusual. Pain, bloating, and rectal urgency are common, and incontinence may occur. Painless diarrhea is not typical.

Diagnosis is based on characteristic bowel patterns, time and character of pain, and exclusion of other disease processes through physical examination and routine diagnostic tests. Diagnostic testing should be more intensive when “red flags” are present: older age, weight loss, rectal bleeding, vomiting. Proctosigmoidoscopy with a flexible fiberoptic instrument should be performed. Introduction of the sigmoidoscope and air insufflation frequently trigger bowel spasm and pain. The mucosal and vascular patterns in IBS usually appear normal. Colonoscopy is preferred for patients >40 with a change in bowel habits, particularly those with no previous IBS symptoms, to exclude colonic polyps and tumors. In patients with chronic diarrhea, particularly older women, mucosal biopsy can rule out possible microscopic colitis.

Paracellular Transport.

Paracellular transport refers to the transfer of substances between cells of an epithelium. It is in contrast to “transcellular transport”, where the substances travel through the cell, passing through both the apical membrane and basolateral membrane. The epithelial lining of luminal organs such as the gastrointestinal tract form a regulated, selectively permeable barrier between luminal contents and the underlying tissue compartments. Paracellular permeability across epithelial and endothelial cells is in large part regulated by an apical intercellular junction also referred to as the tight junction. The tight junction and its subjacent adherens junction constitute the apical junctional complex. Stimuli such as nutrients, internal signaling molecules and cytokines influence the apical F-actin organization and also modulate the AJC structure and paracellular permeability.

### Screening Methods

In other embodiments of the invention, assays are provided to identify candidate agents that act on TG2 activation, including high throughput in vitro cellular or cell-free assays. In the assays of the invention, TG2 is activated by enterocytes treated with γ-IFN. The level of TG2 activity can be monitored by methods known in the art, e.g. by determining the cross-linking of a TG2 substrate. The level of active enzyme may be compared to the total TG2 concentration, e.g. as determined by a suitable affinity assay, etc. Candidate agents may be brought in contact with the system, and the effect on TG2 activation determined after incubation for a period of time sufficient to measure activation where present. As controls, the assay may be compared to the activity on the absence of an agent, or in the presence of an agent shown herein to inhibit TG2 activation, e.g. inhibitors of PI3 kinase, inhibitors of thioredoxin, etc. Cell-free assays may utilize preparations of TG2 in the presence of thioredoxin and upon exposure to buffers with varying redox potentials, where the determination of TG2 activity is as described above. Candidate agents include, without limitation, inhibitors of γ-IFN, inhibitors of PI3 kinase, inhibitors of thioredoxin, inhibitors of TG2, and the like.

The term “agent” as used herein describes any molecule, e.g. protein or pharmaceutical, with the capability of altering TG2 activation or paracellular transport. Generally, a plurality of assay mixtures are run in parallel with different agent concentrations to obtain a differential response to the various concentrations. Typically one of these concentrations serves as a negative control, i.e., at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40. degree. C. Incubation periods are selected for optimum activity, but may also be optimized to facilitate rapid high-throughput screening. Typically between 0.1 and 1 hours will be sufficient.

The compounds having the desired pharmacological activity may be administered in a physiologically acceptable carrier to a host. The inhibitory agents may be administered in a variety of ways, orally, topically, parenterally e.g. subcutaneously, intraperitoneally, by viral infection, intravascularly, etc. Depending upon the manner of introduction, the compounds may be formulated in a variety of ways. The concentration of therapeutically active compound in the formulation may vary from about 0.1-10 wt %.

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature, and the like), but some experimental errors and deviations may be present. Unless indicated otherwise, parts are parts by weight, molecular weight is weight average molecular weight, temperature is in degrees Centigrade, and pressure is at or near atmospheric.

### Example 1

Because IFN-γ is the primary pro-inflammatory cytokine secreted by these T cells, we hypothesized the existence of a signal transduction pathway for extracellular TG2 activation, one that is induced by IFN-γ. To test this hypothesis, we investigated the human intestinal epithelial cell line T84, because of a large body of evidence suggesting that these cells were responsive to IFN-γ. In particular, when the basolateral side of a cultured monolayer of T84 cells is exposed to IFN-γ, its permeability increases, as measured by the trans-epithelial flux of gluten peptides. Using this assay, we have verified the existence of a signal transduction pathway for extracellular TG2 activation, and have also identified PI3 kinase as a promising target for celiac sprue therapy.

IFN-γ Mediated Peptide Flux Across T84 Monolayers is Dominated by Paracellular Transport:

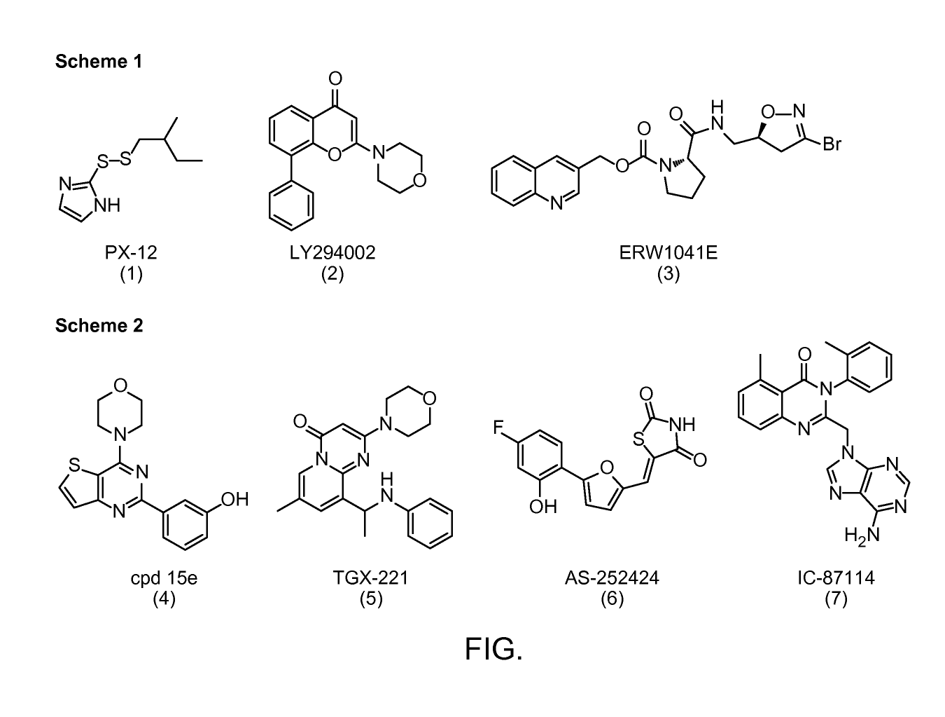

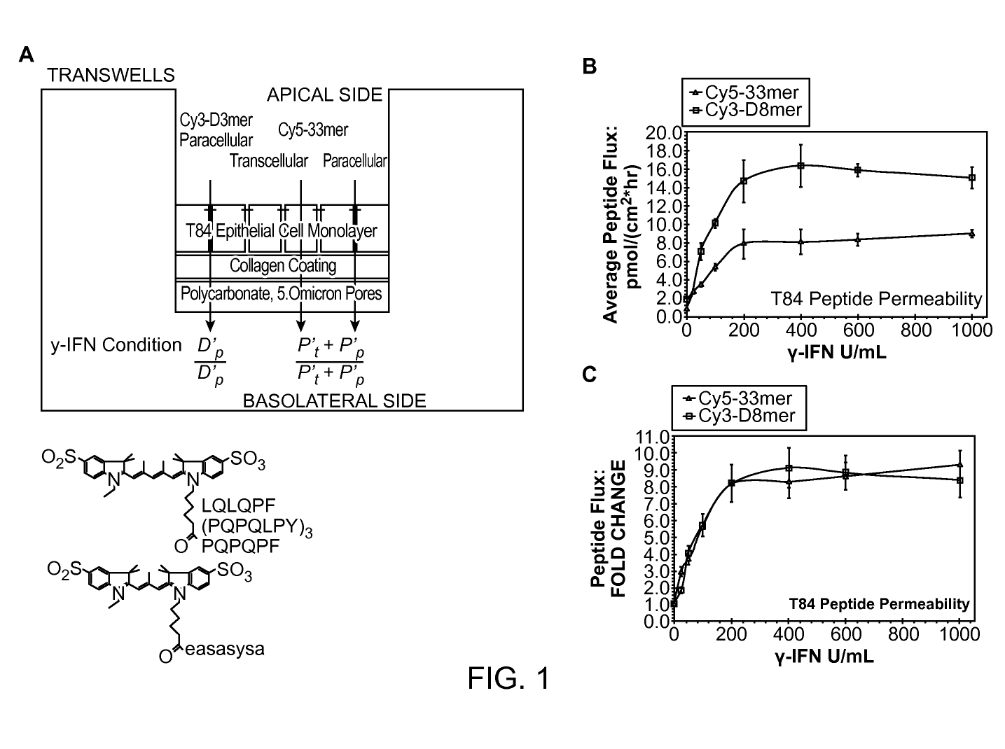

When the T84 model used in this study was exposed to IFN-γ, its trans-epithelial peptide flux increased (FIG. 1A). Specifically, we measured the flux of a Cy5-labeled gluten peptide (LQLQPFPQPQLPYPQPQLPYPQPQLPYPQPQPF, a.k.a. 33mer; as well as a Cy3-labeled octapeptide comprised of D-amino acids that is exclusively transported across the intestinal epithelium via the paracellular route (easasysa, a.k.a. D8mer). T84 cells, grown to maturity on collagen-coated supports, were treated with IFN-γ on the basolateral side for 48 h. The apical-to-basolateral flux of Cy5-33mer and Cy3-D8mer was quantified by sampling the basolateral chamber every hour for 4 h. IFN-γ increased the flux of Cy5-33mer and Cy3-D8mer peptides across the T84 monolayer by as much as 10-fold (FIG. 1B). Within experimental error, this increase in flux was identical for both peptides (FIG. 1C), implying that paracellular transport is the dominant pathway for gluten peptide translocation across the T84 epithelial cell monolayer. In the context of celiac sprue, paracellular peptide transport allows gluten peptides to gain access to TG2 in the extracellular matrix of the small intestine.

IFN-γ Activates TG2 in the Extracellular Matrix of T84 Monolayers:

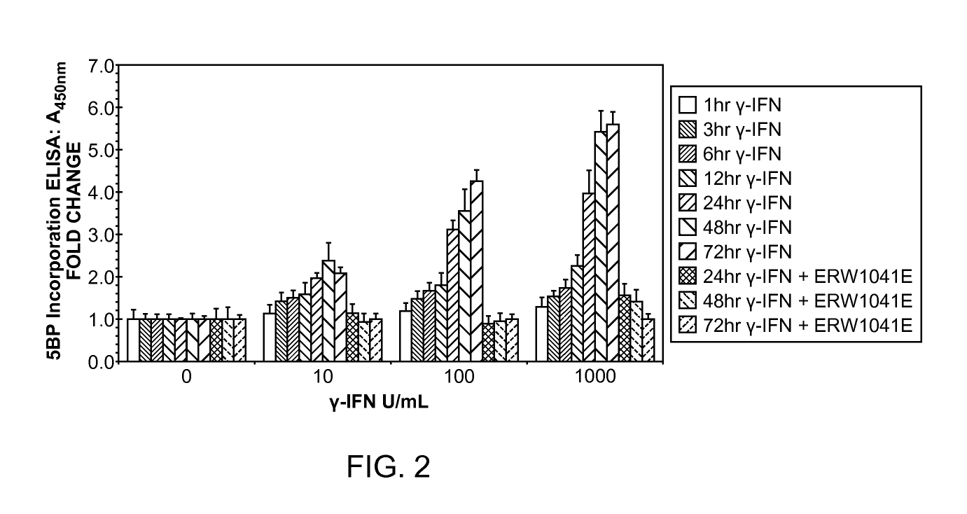

In addition to increasing the paracellular permeability of antigenic gluten peptides, we sought to determine if IFN-γ could activate TG2 in the extracellular matrix of mature T84 monolayers. Again, the basolateral side of these cells was exposed to varying doses of IFN-γ for 48 h. Thereafter, a small molecule substrate of TG2,5-biotinamido pentylamine (5BP), was briefly added to the cell culture medium. During this period, catalytically active TG2 attached 5BP to proteins such as fibronectin in the extracellular matrix. To quantify the extent to which IFN-γ activates TG2 in the T84 model system, an enzyme linked immunosorbent assay (ELISA) was developed, using streptavidin conjugated to horseradish peroxidase. T84 monolayers were exposed to IFN-γ for 1 to 72 h, followed by incubation with 5BP for 4 h. The cultures were then washed with PBS, fixed with 4% (w/v) paraformaldehyde, washed again, and blocked with 5% (w/v) BSA. T84 cells were not permeabilized in this experiment, so that streptavidin recognition would be limited to biotin that is attached to the cell surface or to extracellular matrix proteins. As shown in FIG. 2, TG2 activity increases steadily in response to IFN-γ exposure. Pre-treatment with TG2 inhibitor ERW1041E (3) completely blocked 5BP incorporation, verifying the utility of this cell culture assay for screening candidate TG2 inhibitors that had been identified through biochemical assays.

Role of PI3 Kinase in IFN-γ Mediated TG2 Activation:

Kinases are a promising class of drug targets in the treatment of a variety of human diseases. A number of kinases are thought to influence the barrier function of the T84 intestinal epithelial cell line. Examples include adenosine monophosphate activated protein kinase (AMPK), rho-associated protein kinase (ROCK), serine-threonine protein kinase (AKT), myosin light chain kinase (MLCK), protein kinase C(PKC), and phosphatidylinositide-3-kinase (PI3K) (see McKay et al. (2007) Journal of Pharmacology and Experimental Therapeutics 320: 1013-1022; Choudhury (2004) Journal of Biological Chemistry 279: 27399-27409; and Hwang et al. (2004) Biochemical and Biophysical Research Communications 318: 691-697).

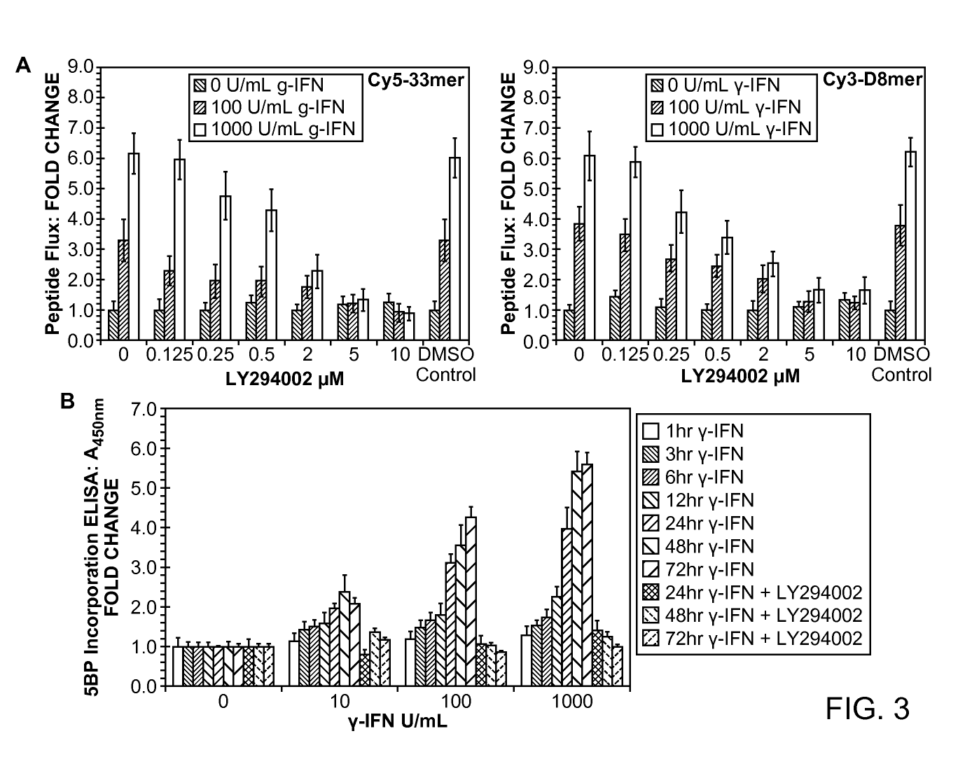

We therefore sought to establish whether a single kinase inhibitor could fully reverse TG2 activation in response to IFN-γ. As shown in FIG. 3, the PI3K inhibitor LY294002 (2) fully negates IFN-γ induced increases in trans-epithelial peptide flux as well as TG2 activity. Both responses to IFN-γ are relevant to celiac disease. Although each has been considered as a promising target for non-dietary therapy, until now they have been regarded as independent consequences of consuming gluten. Our findings establish a mechanistic link between these two phenomena. They also suggest that, LY294002 and, by inference, other PI3K inhibitors are particularly attractive agents for celiac therapy, because a single drug can block both adverse effects of dietary gluten. Scheme 2 illustrates other examples of potentially promising PI3K inhibitors include compound 15e (4) [48], TGX-221 (5) [49], AS-252424 (6) [50], and IC-87114 (7) [51]. Several of these compounds have already been dosed to humans at high doses without serious adverse effects (see Hayakawa et al. (2006) Bioorganic & Medicinal Chemistry 14: 6847-6858; Jackson et al. (2005) Nature Medicine 11: 507-514; Pomel et al. (2006) Journal of Medicinal Chemistry 49: 3857-3871; Sadhu et al. (2003) Journal of Immunology 170: 2647-2654).

Role of Redox Potential in the Activation of Human TG2:

To understand the precise mechanism by which extracellular TG2 activity is induced in response to IFN-γ, we hypothesized that the PI3K signal cascade modulates the redox state of a vicinal disulfide bond that inactivates calcium-bound TG2. Calcium ions and guanine nucleotides are two well-known allosteric regulators of mammalian TG2 activity. In the presence of calcium and the absence of guanine nucleotides, TG2 adopts an, “open” active conformation. Conversely, in the absence of calcium and the presence of guanine nucleotides, TG2 assumes a “closed”, catalytically inactive conformation. Recent studies have revealed a third allosteric regulatory mechanism. Specifically, the formation of a vicinal disulfide bond in the open conformation of the protein reversibly inhibits its enzyme activity. Guided by the assumption that a vast majority of extracellular TG2 exists in this inactive state, we sought to establish whether this disulfide bond could be modulated by IFN-γ, and if so, how.

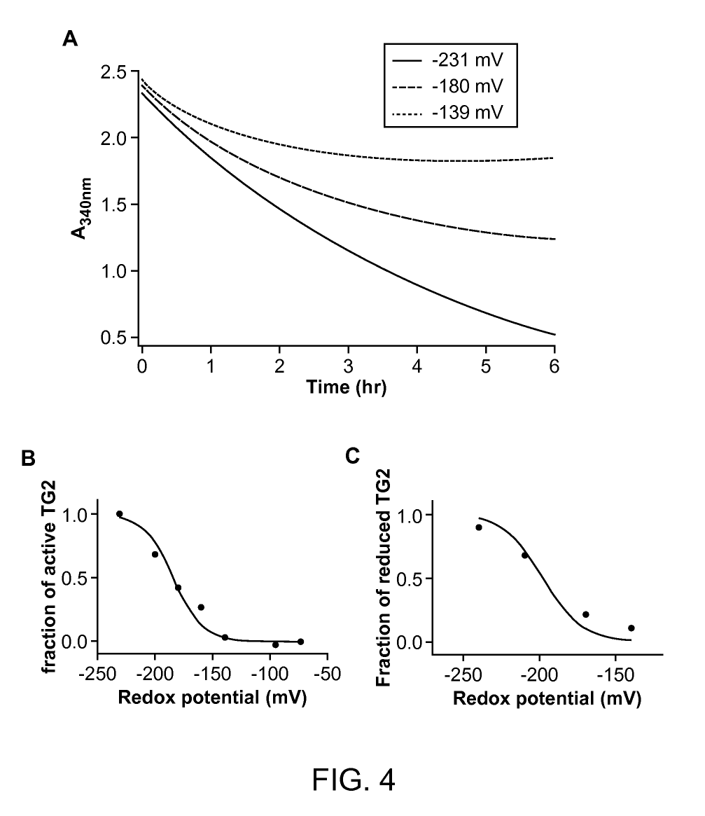

We first sought to measure the redox potential of the vicinal disulfide bond in human TG2. Purified, active TG2 was pre-equilibrated with a 10 mM GSH/GSSG redox buffer over a wide potential range (−70 to −230 mV) for 1 h. The enzymatic activity of the resulting protein was measured in the same redox buffer. The GSH/GSSG ratio was unchanged over the course of activity assay, as judged by analytical HPLC. As expected, TG2 was inactivated when the redox potential increased, although the kinetics of inactivation appeared to be relatively slow (FIG. 4A). Because enzymatic activity attained steady state only after ca. 4 h, the slopes of individual reaction progress curves were calculated from 4-5 h data and compared to the activity of DTT-treated TG2 (FIG. 4B). The redox potential E₀ of TG2 was calculated by fitting the resulting plot to the Nernst equation for a two-electron process

$$( E = {E}_{0} - 29.6 mV \times {log}_{10} \frac{[ redTG 2 ]}{[ oxTG 2 ]} ) ,$$

and was determined to be −184±4 mV.

In an alternative, more direct experiment designed to estimate the E₀ of the vicinal disulfide bond, recombinant human TG2 was pre-equilibrated in appropriate redox buffers for 4 h, after which all of its free cysteine residues were covalently labeled by iodoacetamide. The denatured protein was digested with trypsin, separated by C₁₈ liquid chromatography, and analyzed by ESI mass spectroscopy. From a quantitative analysis of the relative alkylation of C370, C371 and C230, the fractional oxidation state of TG2 under was deduced. In turn, this data was fitted to the same two-electron Nernst equation as above to calculate E₀. The results (E₀=−198±5 mV, FIG. 4C) were in good agreement with the redox potential measured by the activity assay.

In Vitro Activation of Oxidized TG2 by Thioredoxin:

The unusually high redox potential of the vicinal disulfide bond in oxTG2 was entirely consistent with earlier observations that TG2 was predominantly inactive in the extracellular matrix, and suggested that the enzyme may be a critical sensor for subtle decreases in the redox potential of the extracellular matrix. However, even under thermodynamically favorable conditions, the rates of disulfide bond formation/reduction in human TG2 were relatively slow (for example, see FIG. 4A.) We therefore hypothesized that oxidized TG2 in the extracellular matrix was activated through a specific molecular recognition event involving another redox-active protein.

In theory, any disulfide bond reducing agent with an E₀ value lower than −184 mV could activate oxidized TG2. For several reasons, we targeted thioredoxin as such a candidate. First, thioredoxin (Trx) has a much lower E₀ value (−230 mV) than TG2 [53], and could therefore be expected to provide an adequate driving force for the reaction. Second, although Trx is predominantly a cytosolic protein in mammals, it can be found at appreciable concentrations (1-10 nM) in extracellular fluids such as plasma. Last but not least, the plasma levels of Trx are known to undergo significant increases in response to various disease states, a phenomenon that has also motivated clinical targeting of this extracellular protein for the treatment of cancer.

To test our hypothesis, we measured the kinetics of Trx-mediated activation of oxidized TG2. Reduced forms of Trx from E. coli and human were prepared by overexpression in and purification from recombinant E. coli. Dithiothreitol (DTT) was used as a reference small molecule reducing agent. Under comparable conditions, recombinant human Trx reduced oxidized TG2 with a second order rate constant that was at least 100 times higher than DTT, and at least 150 times higher than E. coli Trx. We therefore quantified the specificity of human Trx for human oxidized TG2 by measuring the Michaelis-Menten parameters for Trx-mediated reduction of oxidized TG2. Insulin, a well-characterized extracellular substrate of Trx, was used as a reference, and human thioredoxin reductase was used as a catalyst to achieve turnover under steady-state conditions. The k_{cat}/K_{M} of Trx for insulin and oxidized TG2 were 3.6 μM⁻¹ min⁻¹ and 1.6 μM⁻¹ min⁻¹, respectively, and the K_{M} values were 30 μM and 21 μM, respectively. Based on these parameters, one can estimate that as little as 2.5 nM Trx in the extracellular matrix should be able to activate 10% of the local oxidized TG2 within 30 min.

The small molecule PX-12 (1,1-methylpropyl 2-imidazolyl disulfide) inhibits Trx by oxidizing its active site Cys. In our assay for Trx-mediated activation of oxidized TG2, PX-12 blocked TG2 activation in a dose-dependent fashion, with complete inactivation by 100 μM PX-12. High doses of PX-12 have been administered to humans without any serious adverse effects, suggesting that inhibition of extracellular Trx would be a safe therapeutic option.

Interferon-7 Triggers Trx Secretion and Activates Extracellular TG2:

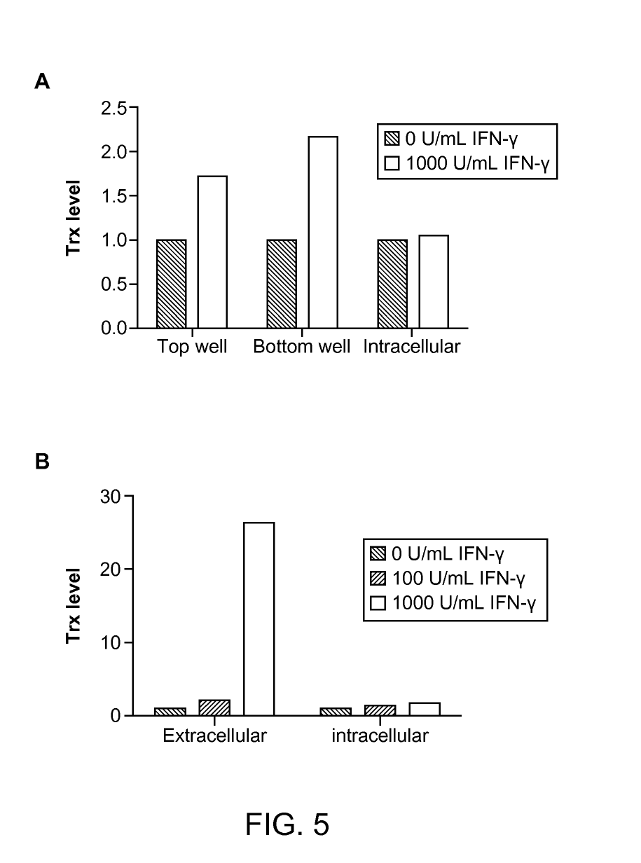

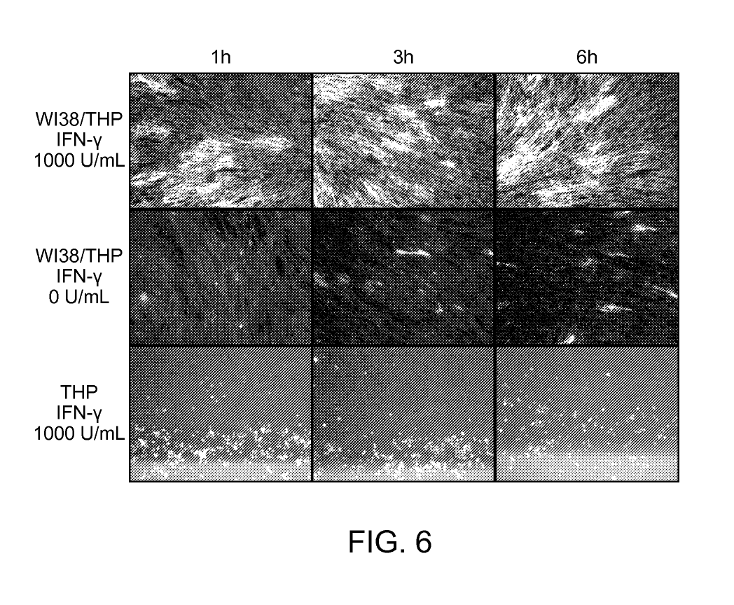

To test whether IFN-γ triggers extracellular TG2 activation via Trx secretion, we used two unrelated assays systems. First, T84 monolayers were used, because their ability to activate TG2 in response to this cytokine had already been quantitatively characterized (see above). We therefore sought to assess whether IFN-γ exposure altered the levels of extracellular Trx in cultured T84 monolayers. As shown in FIG. 5A, Trx secretion was significantly higher on both the apical and basolateral sides of T84 monolayers exposed to IFN-γ. Motivated by an earlier report [61], we also studied the monocytic cell line THP-1 in co-culture with WI-38 fibroblasts. Extracellular Trx in THP-1 cells increased by ca. 30-fold in response to IFN-γ exposure (FIG. 5B). When THP-1 cells pretreated with IFN-γ were co-incubated with WI-38 monolayers for 48 h, strong TG2 activity could be detected around a subset of the fibroblasts (FIG. 6). In the absence of IFN-γ treatment, no TG2 activity was observed. Our findings demonstrate that extracellular TG2 can be efficiently activated by Trx that is secreted in response to IFN-γ. Thus, pharmacological inhibition of extracellular Trx by drug candidates such as PX-12 is expected be an effective strategy for non-dietary therapy of celiac sprue.

Recombinant Human Thioredoxin Activates Extracellular TG2 in Both Human Intestinal Epithelial Cells and Human Fibroblasts:

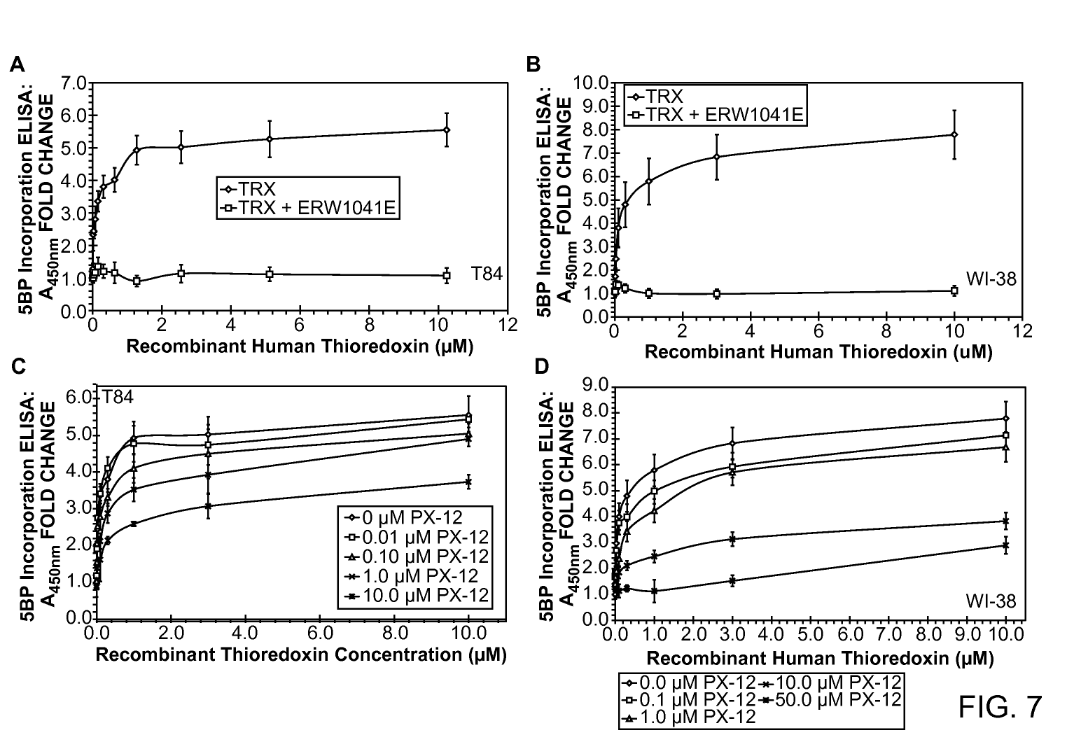

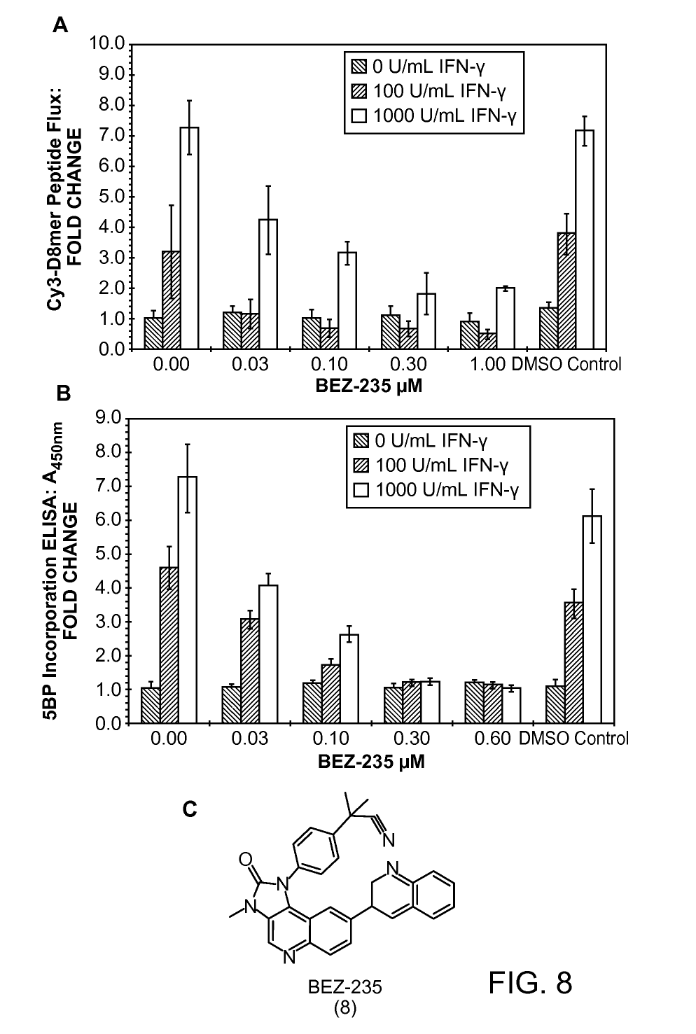

To test whether recombinant human thioredoxin could directly activate TG2 in cell culture, we used T84 and WI-38 monolayers in similar assays as described herein. T84 and WI-38 cells were grown into mature monolayers at which point varying amounts of pre-reduced thioredoxin were added to the culture medium along with 5BP and/or small molecule inhibitors, such as the TG2 inhibitor, ERW1041E, or the thioredoxin inhibitor, PX-12. 5BP incorporated into the extracellular matrix of cultured monolayers via activated TG2 was then quantified. FIG. 7A-B demonstrates the ability of recombinant human thioredoxin to directly influence the activity of extracellular TG2 via increased 5BP incorporation in both T84 and WI-38 monolayers. Furthermore, the addition of the selective TG2 inhibitor, ERW1041E, (FIG. 7A-B) completely negates the thioredoxin induced 5BP incorporation. Lastly, the addition of the known thioredoxin inhibitor PX-12 quantitatively reduced the amount of 5BP incorporation in response to thioredoxin in both cell lines tested (FIG. 7C-D).

These and other methods of the invention can be practiced using the methods provided by the invention.

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

1. A method of reducing tissue transglutaminase (TG2) activation in an individual, the method comprising:
administering to said individual an agent that blocks TG2 activation or activity in a dose effective to provide for a reduction in TG2 activity.

2. The method of claim 1, wherein the TG2 activity is enteric TG2 activity.

3. The method of claim 2, where the individual has an inflammatory enteric disorder.

4. The method of claim 3, wherein the inflammatory enteric disorder is selected from celiac sprue, dermatitis herpetiformis, irritable bowel syndrome and Crohn's Disease.

5. The method of claim 1, wherein the agent inhibits PI3 kinase.

6. The method of claim 5, wherein the agent is LY294002.

7. The method of claim 1, wherein the agent inhibits thioredoxin.

8. The method of claim 7, wherein the agent is selected from the compounds set forth in Table 1.

9. The method of claim 8, wherein the agent is selected from the group consisting of 2-(sec-butyldisulfanyl)-5-nitro-1H-benzo[d]imidazole; 2-(sec-butyldisulfanyl)benzo[d]thiazole; 2-(sec-butyldisulfanyl)benzo[d]oxazole; 2-(cyclopentyldisulfanyl)-1H-benzo[d]imidazole; and 2-(cyclohexyldisulfanyl)-1H-benzo[d]imidazole.

10. The method of claim 1, wherein the agent inhibits TG2.

11. The method of claim 10, wherein the agent is selected from the compound set forth in Table 3 and Table 4.

12. The method of claim 11, wherein the agent is selected from the group consisting of (2S,4S)-quinolin-3-ylmethyl 2-(((S)-3-bromo-4,5-dihydroisoxazol-5-yl)methylcarbamoyl)-4-fluoropyrrolidine-1-carboxylate; (2S,4S)-quinolin-3-ylmethyl 2-(((S)-3-bromo-4,5-dihydroisoxazol-5-yl)methylcarbamoyl)-4-hydroxypyrrolidine-1-carboxylate; (2S,4R)-quinolin-3-ylmethyl 2-(((S)-3-bromo-4,5-dihydroisoxazol-5-yl)methylcarbamoyl)-4-(prop-2-ynyloxy)pyrrolidine-1-carboxylate

13. (canceled)

14. The method of claim 1, wherein the agent is administered orally and is active in the intestine.

15. The method of claim 14, wherein the agent is contained in a formulation that comprises an enteric coating.

16-21. (canceled)

22. A pharmaceutical formulation comprising an effective dose of a compound for inhibition of TG2 activation, wherein the compound is set forth in Table 1.

23. The formulation of claim 22, wherein the agent is selected from the group consisting of 2-(sec-butyldisulfanyl)-5-nitro-1H-benzo[d]imidazole; 2-(sec-butyldisulfanyl)benzo[d]thiazole; 2-(sec-butyldisulfanyl)benzo[d]oxazole; 2-(cyclopentyldisulfanyl)-1H-benzo[d]imidazole; and 2-(cyclohexyldisulfanyl)-1H-benzo[d]imidazole.

24. A pharmaceutical formulation comprising an effective dose of a compound for inhibition of TG2, wherein the compound is set forth in Table 3 and Table 4, wherein the compounds is other than compound (2).

25. The formulation of claim 24, wherein the agent is selected from the group consisting of (2S,4S)-quinolin-3-ylmethyl 2-(((S)-3-bromo-4,5-dihydroisoxazol-5-yl)methylcarbamoyl)-4-fluoropyrrolidine-1-carboxylate; (2S,4S)-quinolin-3-ylmethyl 2-(((S)-3-bromo-4,5-dihydroisoxazol-5-yl)methylcarbamoyl)-4-hydroxypyrrolidine-1-carboxylate; (2S,4R)-quinolin-3-ylmethyl 2-(((S)-3-bromo-4,5-dihydroisoxazol-5-yl)methylcarbamoyl)-4-(prop-2-ynyloxy)pyrrolidine-1-carboxylate.

26-29. (canceled)
